SEQUENCE LISTING (63 sequences)

SEQ ID NO: 1 - protein, 329 aa, Homo sapiens
YTFVLMPINASNALTLAHQLRVYTAEAASFSDMMDVVYVAQMIQKFLGYVDQIKELVEVMVDMASNLMLVDEHLLWLAQREDKACSRIVGALERIGGAALSPHAQHISVNARNVALEAYLIKPHSYVGLTCTAFQRREGGVPGTRPGSPGQNPPPEPEPPADQQLRFRCTTGRPNVSLSSFHIKNSVALASIQLPPSLFSSLPAALAPPVPPDCTLQLLVFRNGRLFHSHSNTSRPGAAGPGKRRGVATPVIFAGTSGCGVGNLTEPVAVSLRHWAEGAEPVAAWWSQEGPGEAGGWTSEGCQLRSSQPNVSALHCQHLGNVAVLMELS

SEQ ID NO: 2 - protein, 334 aa, Homo sapiens
ANDVTRVLYMFNQMPLNLTNAVATARQLLAYTVEAANFSDKMDVIFVAEMIEKFGRFTKEEKSKELGDVMVDIASNIMLADERVLWLAQREAKACSRIVQCLQRIATYRLAGGAHVYSTYSPNIALEAYVIKSTGFTGMTCTVFQKVAASDRTGLSDYGRRDPEGNLDKQLSFKCNVSNTFSSLALKNTIVEASIQLPPSLFSPKQKRELRPTDDSLYKLQLIAFRNGKLFPATGNSTNLADDGKRRTVVTPVILTKIDGVNVDTHHIPVNVTLRRIAHGADAVAARWDFDLLNGQGGWKSDGCHILYSDENITTIQCYSLSNYAVLMDLTGSE

SEQ ID NO: 3 - protein, 303 aa, Homo sapiens
IDYRNIQMMTREHLAKAQRGLPGEGVSEVIQTLVEISQDGTSYSGDLLSTIDVLRNMTEIFRRAYYSPTPGDVQNFVQILSNLLAEENRDKWEEAQLAGPNAKELFRLVEDFVDVIGFRMKDLRDAYQVTDNLVLSIHKLPASGATDISFPMKGWRATGDWAKVPEDRVTVSKSVFSTGLTEADEASVFVVGTVLYRNLGSFLALQRNTTVLNSKVISVTVKPPPRSLRTPLEIEFAHMYNGTTNQTCILWDETDVPSSSAPPQLGPWSWRGCRTVPLDALRTRCLCDRLSTFAILAQLSADA

SEQ ID NO: 4 - protein, 335 aa, Homo sapiens
HEYRYLYLSLREHLAKGQRMLAGEGMSQVVRSLQELLARRTYYSGDLLFSVDILRNVTDTFKRATYVPSADDVQRFFQVVSFMVDAENKEKWDDAQQVSPGSVHLLRVVEDFIHLVGDALKAFQSSLIVTDNLVISIQREPVSAVSSDITFPMRGRRGMKDWVRHSEDRLFLPKEVLSLSSPGKPATSGAAGSPGRGRGPGTVPPGPGHSHQRLLPADPDESSYFVIGAVLYRTLGLILPPPRPPLAVTSRVMTVTVRPPTQPPAEPLITVELSYIINGTTDPHCASWDYSRADASSGDWDTENCQTLETQAAHTRCQCQHLSTFAVLAQPPKDL

SEQ ID NO: 5 - protein, 299 aa, Homo sapiens
NEYRHLQHSIKEHLAKGQRMLAGDGMSQVTKTLLDLTQRKNFYAGDLLMSVEILRNVTDTFKRASYIPASDGVQNFFQIVSNLLDEENKEKWEDAQQIYPGSIELMQVIEDFIHIVGMGMMDFQNSYLMTGNVVASIQKLPAASVLTDINFPMKGRKGMVDWARNSEDRVVIPKSIFTPVSSKELDESSVFVLGAVLYKNLDLILPTLRNYTVINSKIIVVTIRPEPKTTDSFLEIELAHLANGTLNPYCVLWDDSKTNESLGTWSTQGCKTVLTDASHTKCLCDRLSTFAILAQQPRE

SEQ ID NO: 6 - protein, 352 aa, Homo sapiens
VDLRAMNEKLSRNETQVDGARALQLVRALRSATQHTGTLFGNDVRTAYQLLGHVLQHESWQQGFDLAATQDADFHEDVIHSGSALLAPATRAAWEQIQRSEGGTAQLLRRLEGYFSNVARNVRRTYLRPFVIVTANMILAVDIFDKFNFTGARVPRFDTIHEEFPRELESSVSFPADFFRPPEEKEGPLLRPAGRRTTPQTTRPGPGTEREAPISRRRRHPDDAGQFAVALVIIYRTLGQLLPERYDPDRRSLRLPHRPIINTPMVSTLVYSEGAPLPRPLERPVLVEFALLEVEERTKPVCVFWNHSLAVGGTGGWSARGCELLSRNRTHVACQCSHTASFAVLMDISRRE

SEQ ID NO: 7 - protein, 339 aa, Homo sapiens
SELKGFAERLQRNESGLDSGRSQQLALLLRNATQHTAGYFGSDVKVAYQLATRLLAHESTQRGFGLSATQDVHFTENLLRVGSALLDTANKRHWELIQQTEGGTAWLLQHYEAYASALAQNMRHTYLSPFTIVTPNIVISVVRLDKGNFAGAKLPRYEALRGEQPPDLETTVILPESVFRETPPVVRPAGPGEAQEPEELARRQRRHPELSQGEAVASVIIYRTLAGLLPHNYDPDKRSLRVPKRPIINTPVVSISVHDDEELLPRALDKPVTVQFRLLETEERTKPICVFWNHSILVSGTGGWSARGCEVVFRNESHVSCQCNHMTSFAVLMDVSRRE

SEQ ID NO: 8 - protein, 347 aa, Homo sapiens
RELSLLLDGLELNKTALDTMEAKKLAQRLREVTGHTDHYFSQDVRVTARLLAHLLAFESHQQGFGLTATQDAHFNENLLWAGSALLAPETGDLWAALGQRAPGGSPGSAGLVRHLEEYAATLARNMELTYLNPMGLVTPNIMLSIDRMEHPSSPRGARRYPRYHSNLFRGQDAWDPHTHVLLPSQSPRPSPSEVLPTSSSIENSTTSSVVPPPAPPEPEPGISIIILLVYRTLGGLLPAQFQAERRGARLPQNPVMNSPVVSVAVFHGRNFLRGILESPISLEFRLLQTANRSKAICVQWDPPGLAEQHGVWTARDCELVHRNGSHARCRCSRTGTFGVLMDASPRE

SEQ ID NO: 9 - protein, 299 aa, Homo sapiens
LPSLFMTSTASPVMPTDAYHPIITNLTEERKTFQSPGVILSYLQNVSLSLPSKSLSEQTALNLTKTFLKAVGEILLLPGWIALSEDSAVVLSLIDTIDTVMGHVSSNLHGSTPQVTVEGSSAMAEFSVAKILPKTVNSSHYRFPAHGQSFIQIPHEAFHRHAWSTVVGLLYHSMHYYLNNIWPAHTKIAEAMHHQDCLLFATSHLISLEVSPPPTLSQNLSGSPLITVHLKHRLTRKQHSEATNSSNRVFVYCAFLDFSSGEGVWSNHGCALTRGNLTYSVCRCTHLTNFAILMQVVPL

SEQ ID NO: 10 - protein, 271 aa, Homo sapiens
ISRVNALANDIVLLPDPLSEVHGALSPAEASSFLGLLEHVLAMEMAPLGPAALLAVVRFLKRVVALGAGDPELLLTGPWEQLSQGVVSVASLVLEEQVADTWLSLREVIGGPMALVASVQRLAPLLSTSMTSERPRMRIQHRHAGLSGVTVIHSWFTSRVFQHTLEGPDLEPQAPASSEEANRVQRFLSTQVGSAIISSEVWDVTGEVNVAMTFHLQHRAQSPLFPPHPPSPYTGGAWATTGCSVAALYLDSTACFCNHSTSFAILLQIYE

SEQ ID NO: 11 - protein, 282 aa, Homo sapiens
QRVLFKCKEDVIPDNKQIQQCQEGTAVKPAYVSFCAQINNIFSVLDKVCENKTTVVSLKNTTESFVPVLKQISTWTKFTKEETSSLATVFLESVESMTLASFWKPSANITPAVRTEYLDIESKVINKECSEENVTLDLVAKGDKMKIGCSTIEESESTETTGVAFVSFVGMESVLNERFFKDHQAPLTTSEIKLKMNSRVVGGIMTGEKKDGFSDPIIYTLENIQPKQKFERPICVSWSTDVKGGRWTSFGCVILEASETYTICSCNQMANLAVIMASGELT

SEQ ID NO: 12 - protein, 310 aa, Homo sapiens
HQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQ

SEQ ID NO: 13 - protein, 236 aa, Homo sapiens
NTCQDTTSSKTTEGRKELQKIVDKFESLLTNQTLWRTEGRQEISSTATTILRDVESKVLETALKDPEQKVLKIQNDSVAIETQAITDNCSEERKTFNLNVQMNSMDIRCSDIIQGDTQGPSAIAFISYSSLGNIINATFFEEMDKKDQVYLNSQVVSAAIGPKRNVSLSKSVTLTFQHVKMTPSTKKVFCVYWKSTGQGSQWSRDGCFLIHVNKSHTMCNCSHLSSFAVLMALTSQ

SEQ ID NO: 14 - protein, 186 aa, Homo sapiens
MGSRFLLVLLSGASCPPCPKYASCHNSTHCTCEDGFRARSGRTYFHDSSEKCEDINECETGLAKCKYKAYCRNKVGGYICSCLVKYTLFNFLAGIIDYDHPDCYENNSQGTTQSNVDIWVSGVKPGFGKQLPGDKRTKHICVYWEGSEGGWSTEGCSHVHSNGSYTKCKCFHLSSFAVLVALAPKE

SEQ ID NO: 15 - protein, 327 aa, Homo sapiens
SSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHY

SEQ ID NO: 16 - protein, 274 aa, Homo sapiens
SLLEELNKNFSMIVGNATEAAVSSFVQNLSVIIRQNPSTTVGNLASVVSILSNISSLSLASHFRVSNSTMEDVISIADNILNSASVTNWTVLLREEKYASSRLLETLENISTLVPPTALPLNFSRKFIDWKGIPVNKSQLKRGYSYQIKMCPQNTSIPIRGRVLIGSDQFQRSLPETIISMASLTLGNILPVSKNGNAQVNGPVISTVIQNYSINEVFLFFSKIESNLSQPHCVFWDFSHLQWNDAGCHLVNETQDIVTCQCTHLTSFSILMSP

SEQ ID NO: 17 - protein, 293 aa, Homo sapiens
LNALNIFEEDSRLVQPFEDNIKISVYTGKSETITDMLLQKCPTDLSCVIRNIQQSPWIPGNIAVIVQLLHNISTAIWTGVDEAKMQSYSTIANHILNSKSISNWTFIPDRNSSYILLHSVNSFARRLFIDKHPVDISDVFIHTMGTTISGDNIGKNFTFSMRINDTSNEVTGRVLISRDELRKVPSPSQVISIAFPTIGAILEASLLENVTVNGLVLSAILPKELKRISLIFEKISKSEERRTQCVGWHSVENRWDQQACKMIQENSQQAVCKCRPSKLFTSFSILMSPHILE

SEQ ID NO: 18 - protein, 285 aa, Homo sapiens
ARLLALFTRTKLLQAGQGSPAEEVPQILAQLPGQAAEASSPSDLLTLLSTMKYVAKVVAEARIQLDRRALKNLLIATDKVLDMDTRSLWTLAQARKPWAGSTLLLAVETLACSLCPQDHPFAFSLPNVLLQSQLFGPTFPADYSISFPTRPPLQAQIPRHSLAPLVRNGTEISITSLVLRKLDHLLPSNYGQGLGDSLYATPGLVLVISIMAGDRAFSQGEVIMDFGNTDGSPHCVFWDHSLFQGRGGWSKEGCQAQVASASPTAQCLCQHLTAFSVLMSPHTVP

SEQ ID NO: 19 - protein, 305 aa, Homo sapiens
LSVEKLFKDSTGASRLSVAAPSIPLHILDFRAPETIESVAQGIRKNCPFDYACITDMVKSSETTSGNIAFIVELLKNISTDLSDNVTREKMKSYSEVANHILDTAAISNWAFIPNKNASSDLLQSVNLFARQLHIHNNSENIVNELFIQTKGFHINHNTSEKSLNFSMSMNNTTEDILGMVQIPRQELRKLWPNASQAISIAFPTLGAILREAHLQNVSLPRQVNGLVLSVVLPERLQEIILTFEKINKTRNARAQCVGWHSKKRRWDEKACQMMLDIRNEVKCRCNYTSVVMSFSILMSSKSMT

SEQ ID NO: 20 - protein, 277 aa, Homo sapiens
APINSLLQMAKALIKSPSQDEMLPTYLKDLSISIDKAEHEISSSPGSLGAIINILDLLSTVPTQVNSEMMTHVLSTVNVILGKPVLNTWKVLQQQWTNQSSQLLHSVERFSQALQSGDSPPLSFSQTNVQMSSMVIKSSHPETYQQRFVFPYFDLWGNVVIDKSYLENLQSDSSIVTMAFPTLQAILAQDIQENNFAESLVMTTTVSHNTTMPFRISMTFKNNSPSGGETKCVFWNFRLANNTGGWDSSGCYVEEGDGDNVTCICDHLTSFSILMSP

SEQ ID NO: 21 - protein, 266 aa, Homo sapiens
AQGPPLLATSVTSWWSPQNISLPSAASFTFSFHSPPHTAAHNASVDMCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRSGEAEKRLLLVDFSSQALFQDKNSSQVLGEKVLGIVVQNTKVANLTEPVVLTFQHQLQPKNVTLQCVFWVEDPTLSSPGHWSSAGCETVRRETQTSCFCNHLTYFAVLMVSSVEV

SEQ ID NO: 22 - protein, 280 aa, Homo sapiens
PVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTS

SEQ ID NO: 23 - protein, 258 aa, Homo sapiens
MATPRGLGALLLLLLLPTSGQEKPTEGPRNTCLGSNNMYDIFNLNDKALCFTKCRQSGSDSCNVENLQRYWLNYEAHLMKEGLTQKVNTPFLKALVQNLSTNTAEDFYFSLEPSQVPRQVMKDEDKPPDRVRLPKSLFRSLPGNRSVVRLAVTILDIGPGTLFKGPRLGLGDGSGVLNNRLVGLSVGQMHVTKLAEPLEIVFSHQRPPPNMTLTCVFWDVTKGTTGDWSSEGCSTEVRPEGTVCCCDHLTFFALLLRP

SEQ ID NO: 24 - protein, 298 aa, Homo sapiens
LQELPDKIVDLANITISDENAEDVAEHILNLINESPALGKEETKIIVSKISDISQCDEISMNLTHVMLQIINVVLEKQNNSASDLHEISNEILRIIERTGHKMEFSGQIANLTVAGLALAVLRGDHTFDGMAFSIHSYEEGTDPEIFLGNVPVGGILASIYLPKSLTERIPLSNLQTILFNFFGQTSLFKTKNVTKALTTYVVSASISDDMFIQNLADPVVITLQHIGGNQNYGQVHCAFWDFENNNGLGGWNSSGCKVKETNVNYTICQCDHLTHFGVLMDLSRSTVDSVNEQILAL

SEQ ID NO: 25 - protein, 235 aa, Homo sapiens
MDHCGALFLCLCLLTLQNATTETWEELLSYMENMQVSRGRSSVFSSRQLHQLEQMLLNTSFPGYNLTLQTPTIQSLAFKLSCDFSGLSLTSATLKRVPQAGGQHARGQHAMQFPAELTRDACKTRPRELRLICIYFSNTHFFKDENNSSLLNNYVLGAQLSHGHVNNLRDPVNISFWHNQSLEGYTLTCVFWKEGARKQPWGGWSPEGCRTEQPSHSQVLCRCNHLTYFAVLMQL

SEQ ID NO: 26 - protein, 273 aa, Homo sapiens
LKEANEVANQILNLTADGQNLTSANITNIVEQVKRIVNKEENIDITLGSTLMNIFSNILSSSDSDLLESSSEALKTIDELAFKIDLNSTSHVNITTRNLALSVSSLLPGTNAISNFSIGLPSNNESYFQMDFESGQVDPLASVILPPNLLENLSPEDSVLVRRAQFTFFNKTGLFQDVGPQRKTLVSYVMACSIGNITIQNLKDPVQIKIKHTRTQEVHHPICAFWDLNKNKSFGGWNTSGCVAHRDSDASETVCLCNHFTHFGVLMDLPRSA

SEQ ID NO: 27 - protein, 268 aa, Homo sapiens
LNNISSEVQILTSDANKLTAENITSATRVVGQIFNTSRNASPEAKKVAIVTVSQLLDASEDAFQRVAATANDDALTTLIEQMETYSLSLGNQSVVEPNIAIQSANFSSENAVGPSNVRFSVQKGASSSLVSSSTFIHTNVDGLNPDAQTELQVLLNMTKNYTKTCGFVVYQNDKLFQSKTFTAKSDFSQKIISSKTDENEQDQSASVDMVFSPKYNQKEFQLYSYACVYWNLSAKDWDTYGCQKDKGTDGFLRCRCNHTTNFAVLMTF

SEQ ID NO: 28 - protein, 316 aa, Homo sapiens
PWVNQVAQKIKSGENAANIASELARHTRGSIYAGDVSSSVKLMEQLLDILDAQLQALRPIERESAGKNYNKMHKRERTCKDYIKAVVETVDNLLRPEALESWKDMNATEQVHTATMLLDVLEEGAFLLADNVREPARFLAAKENVVLEVTVLNTEGQVQELVFPQEEYPRKNSIQLSAKTIKQNSRNGVVKVVFILYNNLGLFLSTENATVKLAGEAGPGGPGGASLVVNSQVIAASINKESSRVFLMDPVIFTVAHLEDKNHFNANCSFWNYSERSMLGYWSTQGCRLVESNKTHTTCACSHLTNFAVLMAHREI

SEQ ID NO: 29 - protein, 313 aa, Homo sapiens
HWVNQLAQKIRSGENAASLANELAKHTKGPVFAGDVSSSVRLMEQLVDILDAQLQELKPSEKDSAGRSYNKLQKREKTCRAYLKAIVDTVDNLLRPEALESWKHMNSSEQAHTATMLLDTLEEGAFVLADNLLEPTRVSMPTENIVLEVAVLSTEGQIQDFKFPLGIKGAGSSIQLSANTVKQNSRNGLAKLVFIIYRSLGQFLSTENATIKLGADFIGRNSTIAVNSHVISVSINKESSRVYLTDPVLFTLPHIDPDNYFNANCSFWNYSERTMMGYWSTQGCKLVDTNKTRTTCACSHLTNFAILMAHREI

SEQ ID NO: 30 - protein, 327 aa, Homo sapiens
PWVNHITQKLKSGETAANIARELAEQTRNHLNAGDITYSVRAMDQLVGLLDVQLRNLTPGGKDSAARSLNKLQKRERSCRAYVQAMVETVNNLLQPQALNAWRDLTTSDQLRAATMLLHTVEESAFVLADNLLKTDIVRENTDNIKLEVARLSTEGNLEDLKFPENMGHGSTIQLSANTLKQNGRNGEIRVAFVLYNNLGPYLSTENASMKLGTEALSTNHSVIVNSPVITAAINKEFSNKVYLADPVVFTVKHIKQSEENFNPNCSFWSYSKRTMTGYWSTQGCRLLTTNKTHTTCSCNHLTNFAVLMAHVEVKHSDAVHDLLLDV

SEQ ID NO: 31 - protein, 296 aa, Homo sapiens
ANINKTLTKIRSIKEPVALLQEVYRNSVTDLSPTDIITYIEILAESSSLLGYKNNTISAKDTLSNSTLTEFVKTVNNFVQRDTFVVWDKLSVNHRRTHLTKLMHTVEQATLRISQSFQKTTEFDTNSTDIALKVFFFDSYNMKHIHPHMNMDGDYINIFPKRKAAYDSNGNVAVAFVYYKSIGPLLSSSDNFLLKPQNYDNSEEEERVISSVISVSMSSNPPTLYELEKITFTLSHRKVTDRYRSLCAFWNYSPDTMNGSWSSEGCELTYSNETHTSCRCNHLTHFAILMSSGPSI

SEQ ID NO: 32 - protein, 278 aa, Homo sapiens
QAIWGLADQLHQPVNDDILNRVLHTISMKVATENTDEQLSAMMHLIEKITTEGKIQAFSVASRTLFYEILCSLINPKRKDTRGFSHFAEVTENFAFSLLTNVTCGSPGEKSKTILDSCPYLSILALHWYPQQINGHKFEGKEGDYIRIPERLLDVQDAEIMAGKSTCKLVQFTEYSSQQWFISGNNLPTLKNKVLSLSVKGQSSQLLTNDNEVLYRIYAAEPRIIPQTSLCLLWNQAAASWLSDSQFCKVVEETADYVECACSHMSVYAVYARTDNLS

SEQ ID NO: 33 - protein, 422 aa, Homo sapiens
AQIRKNITETLVSLRVHTVDDIQQIAAALAQCMGPSRELVCRSCLKQTLHKLEAMMLILQAETTAGTVTPTAIGDSILNITGDLIHLASSDVRAPQPSELGAESPSRMVASQAYNLTSALMRILMRSRVLNEEPLTLAGEEIVAQGKRSDPRSLLCYGGAPGPGCHFSIPEAFSGALANLSDVVQLIFLVDSNPFPFGYISNYTVSTKVASMAFQTQAGAQIPIERLASERAITVKVPNNSDWAARGHRSSANSANSVVVQPQASVGAVVTLDSSNPAAGLHLQLNYTLLDGHYLSEEPEPYLAVYLHSEPRPNEHNCSASRRIRPESLQGADHRPYTFFISPGSRDPAGSYHLNLSSHFRWSALQVSVGLYTSLCQYFSEEDMVWRTEGLLPLEETSPRQAVCLTRHLTAFGASLFVPPSH

SEQ ID NO: 34 - protein, 430 aa, Homo sapiens
NLSTLQLMGSYTEIRNYITVITRILSRLSKEDKTASCNQWSRIQDALISSVCRLAFVDQEEMIGSVLMLRDLVSFSNKLGFMSAVLILKYTRALLAQGQFSGPFVIDKGVRLELIGLISRVWEVSEQENSKEEVYRHEEGITVISDLLLGCLSLNHVSTGQMEFRTLLHYNLQSSVQSLGSVQVHLPGDLAGHSPAGAETQSPCYISQLILFKKNPYPGSQAPGQIGGVVGLNLYTCSSRRPINRQWLRKPVMVEFGEEDGLDNRRNKTTFVLLRDKVNLHQFTELSENPQESLQIEIEFSKPVTRAFPVMLLVRFSEKPTPSDFLVKQIYFWDESIVQIYIPAASQKDASVGYLSLLDADYDRKPPNRYLAKAVNYTVHFQWIRCLFWDKREWKSERFSPQPGTSPEKVNCSYHRLAAFALLRRKLKAS

SEQ ID NO: 35 - protein, 411 aa, Homo sapiens
SLSIDVRQKVREHVLGSLSAVTTGLEDVQRVQELAEVLREVTCRSKELTPSAQWEASLALQHASEALLTVSAKARPEDQRRQAATRDLFQAVGSVLEASLSNRPEEPAEASSSQIATVLRLLRVMEHVQTTLLLGKLPGGLPAMLATPSISVYTNRIQPWSWQGSSLRPDAADSATFMLPAASSLSSLEGGQEPVDIKIMSFPKSPFPARSHFDVSGTVGGLRVTSPSGQLIPVKNLSENIEILLPRHSQRHSQPTVLNLTSPEALWVNVTSGEATLGIQLHWRPDIALTLSLGYGYHPNKSSYDAQTHLVPMVAPDELPTWILSPQDLRFGEGVYYLTVVPESDLEPAPGRDLTVGITTFLSHCVFWDEVQETWDDSGCQVGPRTSPYQTHCLCNHLTFFGSTFLVMSNA

SEQ ID NO: 36 - protein, 411 aa, Homo sapiens
SLQKASGQVIDEIAGNFSRAVHGLQALNKLQEACEFLQKLTALTPRFSKPAQVNLINSLIYLSEELLRIPFQNNNSLGFKVPPTVCPFHSLNNVTKAGEGSWLESKRHTEPVEDILEMSLVEFGNIGEAFLEQNQSPESSVTLTSANATLLLSRQNISTLPLSSYTLGHPAPVRLGFPSALALKELLNKHPGVNVQITGLAFNPFKDLDNRNIVGSIGSVLLSANRKLLQVHDLMEDIEIMLWRNVSLETHPTSLNMSTHQLTITVNVTSLEKSLIVSIDPDSPLLMTLYLGFQYQPNCTHFHLNITLPKDKVWQKDEEYTWVLNPEHLQHGIGTYYITAVLSERQEGAQQTPSLVSVITAVTQCYYWEIHNQTWSSAGCQVGPQSTILRTQCLCNHLTFFASDFFVVPRT

SEQ ID NO: 37 - protein, 439 aa, Homo sapiens
IVASVLNNMKTELPLRDDRVNLRKHLIDQSFLLPVSTLVEIGQVVMTITKLTQKPSEFTWDAQKRATMRVWQANQALQEYQQKDKRFRSEQIEIVSTGILMSLSNILKMTSPHQVVKDPFYVIESLSDTILANKVPGNKTTSMRTPNFNMYVKKVEKWGINQLFRNEKHCRNCFYPTLNVSSVPGLSANGPISTMFCDFTNDLFPWLNDQENTSVEVSGFRMTGVADNGSVLEITPDVAEVYLVRKNLTFAAFNLTVGPNSEVDGSLKKTTGGFSFQVDSTVLREVLVHIVTEVMVLFTVLVYTGSQITPTALVATFLVPHDIPPFASQSALFDPACTVKKARVVCLPVSLLQLIAQHSHSPHCTVSIVLQAPRFVMKLNDKLVRISIFSVQCLDMYGIQSEWREGYCILGEKTSWYEVHCICKNVVRARRQLGTIGLT

SEQ ID NO: 38 - protein, 26 aa, Caenorhabditis elegans
HLTHFAVLMDVRGHDLNEIDQTLLTL

SEQ ID NO: 39 - protein, 26 aa, Rattus norvegicus
HLTNFAVLMAHREIYQGRINELLLSV

SEQ ID NO: 40 - protein, 28 aa, Drosophila melanogaster
HLTNFAILMDVVDEHQHSLFTMFDGNMR

SEQ ID NO: 41 - protein, 24 aa, Homo sapiens
HLTYFAVLMQLSPALVPAELLAPL

SEQ ID NO: 42 - protein, 24 aa, Homo sapiens
HLTYFAVLMVSSVEVDAVHKHYLS

SEQ ID NO: 43 - protein, 26 aa, Homo sapiens
HLTNFAILMQVVPLELARGHQVALSS

SEQ ID NO: 44 - protein, 19 aa, Homo sapiens
HLSSFAILMAHYDVEDWKL

SEQ ID NO: 45 - protein, 26 aa, Homo sapiens
HFTHFGVLMDLPRSASQLDARNTKVL

SEQ ID NO: 46 - protein, 26 aa, Drosophila melanogaster
HISSYAVIVDVIDPEDIPEPSLLVQI

SEQ ID NO: 47 - protein, 26 aa, Homo sapiens
HLTAFGASLFVPPSHVRFVFPEPTAD

SEQ ID NO: 48 - protein, 30 aa, Caenorhabditis elegans
HLTMFSVGAFNPTIDADFSYNYNVNEIEKN

SEQ ID NO: 49 - protein, 30 aa, Homo sapiens
RLAAFALLRRKLKASFEVSDISKLQSHPEN

SEQ ID NO: 50 - protein, 32 aa, Homo sapiens
HLTFFGSTFLVMSNAINIHQTAELFATFEDNP

SEQ ID NO: 51 - protein, 32 aa, Homo sapiens
HLTFFASDFFVVPRTVNVEDTIKLFLRVTNNP

SEQ ID NO: 52 - protein, 69 aa, Homo sapiens
QSATDVAAFLGALASLGSLNIPYKIEAVQSETVEPPPPAQLHFMYVAAAAFVLLFFVGCGVLLSRKRRR

SEQ ID NO: 53 - protein, 64 aa, Homo sapiens
KNTDAAAALLASHAIQGTLSYPLVSVVSESLTPERTQLLYLLAVAVVIILFIILLGVIMAKRKR

SEQ ID NO: 54 - protein, 68 aa, Homo sapiens
PDAQSAADYLGALSAVERLDFPYPLRDVRGEPLEPPEPSVPLLPLLVAGAVLLLVILVLGVMVARRKR

SEQ ID NO: 55 - protein, 74 aa, Homo sapiens
WDPGLLLRFLAAMAAVGALEPLLPGPLLAVHPHAGTAPPANQLPWPVLCSPVAGVILLALGALLVLQLIRRRRR

SEQ ID NO: 56 - protein, 70 aa, Drosophila melanogaster
GNMRLIKIDTAAKHQLRNDFQIHSVRGIKNPGDEDNGEPPANVKYVITGIILVIIALAFFGMVLSTQRKR

SEQ ID NO: 57 - protein, 49 aa, Drosophila melanogaster
IHSVRGIKNPGDEDNGEPPANVKYVITGIILVIIALAFFGMVLSTQRKR

SEQ ID NO: 58 - protein, 45 aa, Mus musculus
IEAVKSEPVEPPLPSQLHLMYVAAAAFVLLFFVGCGVLLSRKRRR

SEQ ID NO: 59 - protein, 1125 aa, Artificial Sequence
  Full-length NTF release sensor for dmCIRL used in Example 2
MKLCILLAVVAFVGLSLGETGFPKSLSVLNSRCAHKQSCGVLAATSMFGDPCPGTHKYLEAHYQCISAAQTSTTTNRPSPPPWVLSNGPPIFGNGSGLIHPPGVGAGAPPPPRLPTLPGVVGISGNPGLFNVPPQHTAVTHSTPSSSTTAVGGGRLKGGATSTTTTKHPAGRHDGLPPPPQLHHHHNHHGEDTASPTKPSSKLPAGGNATSPSNTRILTGVGGSGTDDGTLLTTKSSPNRPPGTAASGSVVPGNGSVVRTINNINLNAAGMSGGDDESKLFCGPTHARNLYWNMTRVGDVNVQPCPGGAAGIAKWRCVLMKRIPDSGYDEYDDDISSTTPAPSGGDCLHNSSSCEPPVSMAHKVNQRLRNFEPTWHPATPDLTQCRSLWLNNLEMRVNQRDSSLISIANDMSEVTSSKTLYGGDMLVTTKIIQTVSEKMMHDKETFPDQRQREAMIMELLHCVVKTGSNLLDESQLSSWLDLNPEDQMRVATSLLTGLEYNAFLLADTIIRERSVVQKVKNILLSVRVLETKTIQSSVVFPDSDQWPLSSDRIELPRAALIDNSEGGLVRIVFAAFDRLESILKPSYDHFDLKSSRSYAILSNDSDVNAGEIQQRLRILNSKVISASLGKGRHIQLSQPITLTLKHLKTENVTNPTCVFWNYIDHAWSANGCSLESTNRTHSVCSCNHLTNFAILMDVVDEHQHSLFTMFDGNMRPRIDTAAKHQLRNDFQIHSVRGIKNPGDEDNGEPPANVKYVITGIILVIIALAFFGMVLSTQRKRSGPPKLKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLEFPGIRRPAGIPGDLAPPTDVSLGDELHLDGEDVAMAHADALDDFDLDMLGDGDSPGPGFTPHDSAPYGALDMADFEFEQMFTDALGIDEYGGASRYLDPAFLYKVVSSATMDYKDHDGDYKDHDIDYKDDDDKHRST

SEQ ID NO: 60 - protein, 1125 aa, Artificial Sequence
  Full-length NTF release sensor for dmCIRL (GPS cleavage site mutated)
MKLCILLAVVAFVGLSLGETGFPKSLSVLNSRCAHKQSCGVLAATSMFGDPCPGTHKYLEAHYQCISAAQTSTTTNRPSPPPWVLSNGPPIFGNGSGLIHPPGVGAGAPPPPRLPTLPGVVGISGNPGLFNVPPQHTAVTHSTPSSSTTAVGGGRLKGGATSTTTTKHPAGRHDGLPPPPQLHHHHNHHGEDTASPTKPSSKLPAGGNATSPSNTRILTGVGGSGTDDGTLLTTKSSPNRPPGTAASGSVVPGNGSVVRTINNINLNAAGMSGGDDESKLFCGPTHARNLYWNMTRVGDVNVQPCPGGAAGIAKWRCVLMKRIPDSGYDEYDDDISSTTPAPSGGDCLHNSSSCEPPVSMAHKVNQRLRNFEPTWHPATPDLTQCRSLWLNNLEMRVNQRDSSLISIANDMSEVTSSKTLYGGDMLVTTKIIQTVSEKMMHDKETFPDQRQREAMIMELLHCVVKTGSNLLDESQLSSWLDLNPEDQMRVATSLLTGLEYNAFLLADTIIRERSVVQKVKNILLSVRVLETKTIQSSVVFPDSDQWPLSSDRIELPRAALIDNSEGGLVRIVFAAFDRLESILKPSYDHFDLKSSRSYAILSNDSDVNAGEIQQRLRILNSKVISASLGKGRHIQLSQPITLTLKHLKTENVTNPTCVFWNYIDHAWSANGCSLESTNRTHSVCSCNALTNFAILMDVVDEHQHSLFTMFDGNMRPRIDTAAKHQLRNDFQIHSVRGIKNPGDEDNGEPPANVKYVITGIILVIIALAFFGMVLSTQRKRSGPPKLKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLEFPGIRRPAGIPGDLAPPTDVSLGDELHLDGEDVAMAHADALDDFDLDMLGDGDSPGPGFTPHDSAPYGALDMADFEFEQMFTDALGIDEYGGASRYLDPAFLYKVVSSATMDYKDHDGDYKDHDIDYKDDDDKHRST

SEQ ID NO: 61 - protein, 1125 aa, Artificial Sequence
  Full-length NTF release sensor for dmCIRL (S3 cleavage site mutated)
MKLCILLAVVAFVGLSLGETGFPKSLSVLNSRCAHKQSCGVLAATSMFGDPCPGTHKYLEAHYQCISAAQTSTTTNRPSPPPWVLSNGPPIFGNGSGLIHPPGVGAGAPPPPRLPTLPGVVGISGNPGLFNVPPQHTAVTHSTPSSSTTAVGGGRLKGGATSTTTTKHPAGRHDGLPPPPQLHHHHNHHGEDTASPTKPSSKLPAGGNATSPSNTRILTGVGGSGTDDGTLLTTKSSPNRPPGTAASGSVVPGNGSVVRTINNINLNAAGMSGGDDESKLFCGPTHARNLYWNMTRVGDVNVQPCPGGAAGIAKWRCVLMKRIPDSGYDEYDDDISSTTPAPSGGDCLHNSSSCEPPVSMAHKVNQRLRNFEPTWHPATPDLTQCRSLWLNNLEMRVNQRDSSLISIANDMSEVTSSKTLYGGDMLVTTKIIQTVSEKMMHDKETFPDQRQREAMIMELLHCVVKTGSNLLDESQLSSWLDLNPEDQMRVATSLLTGLEYNAFLLADTIIRERSVVQKVKNILLSVRVLETKTIQSSVVFPDSDQWPLSSDRIELPRAALIDNSEGGLVRIVFAAFDRLESILKPSYDHFDLKSSRSYAILSNDSDVNAGEIQQRLRILNSKVISASLGKGRHIQLSQPITLTLKHLKTENVTNPTCVFWNYIDHAWSANGCSLESTNRTHSVCSCNHLTNFAILMDVVDEHQHSLFTMFDGNMRPRIDTAAKHQLRNDFQIHSVRGIKNPGDEDNGEPPANVKYVITGIILVIIALAFFGMKLSTQRKRSGPPKLKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLEFPGIRRPAGIPGDLAPPTDVSLGDELHLDGEDVAMAHADALDDFDLDMLGDGDSPGPGFTPHDSAPYGALDMADFEFEQMFTDALGIDEYGGASRYLDPAFLYKVVSSATMDYKDHDGDYKDHDIDYKDDDDKHRST

SEQ ID NO: 62 - protein, 432 aa, Artificial Sequence
  NTF release sensor positive control
MQSQRSRRRSRAPNTWICFWINKMHAVASLPASLPLLLLTLAFANLPNTVRGTDTALVAASCTSVGCQNIDTAAKHQLRNDFQIHSVRGIKNPGDEDNGEPPANVKYVITGIILVIIALAFFGMVLSTQRKRSGPPKLKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLEFPGIRRPAGIPGDLAPPTDVSLGDELHLDGEDVAMAHADALDDFDLDMLGDGDSPGPGFTPHDSAPYGALDMADFEFEQMFTDALGIDEYGG

SEQ ID NO: 63 - protein, 1187 aa, Artificial Sequence
  NTF release sensor negative control
MQSQRSRRRSRAPNTWICFWINKMHAVASLPASLPLLLLTLAFANLPNTVRGTDTALVAASCTSVGCQNGGTCVTQLNGKTYCACDSHYVGDYCEHRNPCNSMRCQNGGTCQVTFRNGHPGISCKCPLGFDESLCEIAVPEDCTESSCLNGGSCIDGINGYNCSCLAGYSGANCQYKLNKCDSNPCLNGATCHEQNNEYTCHCPSGFTGKQCSEYVDWCGQSPCENGATCSQMKHQFSCKCSAGWTGKLCDVQTISCQDAADRKGLSLRQLCNNGTCKDYGNSHVCYCSQGYAGSYCQKEIDECQSQPCQNGGTCRDLIGAYECQCRQGFQGQNCELNIDDCAPNPCQNGGTCHHRVMNFSCSCPPGTMGIICEINKDDCKPGACHNNGSCIDRVGGFECVCQPGFVGARCEGDINECLSNPCSNAGTLDCVQLVNNYHCNCRPGHMGRHCEHKVDFCAQSPCQNGGNCNIRQSGHHCICNNGFYGKNCELSGQDCDSNPCRVGNCVVADEGFGYRCECPRGTLGEHCEIDTLDECSPNPCAQGAACEDLLGDYECLCPSKWKGKRCDIYDANYPGWNGGSGSGNDRYAADLEQQRAMCDKRGCTEKQGNGICDSDCNTYACNFDGNDCSLGINPWANCTANECWNKFKNGKCNEECNNAACHYDGHDCERKLKSCDTLFDAYCQKHYGDGFCDYGCNNAECSWDGLDCENKTQSPVLAEGAMSVVMLMNVEAFREIQAQFLRNMSHMLRTTVRLKKDALGHDIIINWKDNVRVPEIEDTDFARKNKILYTQQVHQTGIQIYLEIDNRKCTECFTHAVEAAEFLAATAAKHQLRNDFQIHSVRGIKNPGDEDNGEPPANVKYVITGIILVIIALAFFGMVLSTQRKRSGPPKLKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLEFPGIRRPAGIPGDLAPPTDVSLGDELHLDGEDVAMAHADALDDFDLDMLGDGDSPGPGFTPHDSAPYGALDMADFEFEQMFTDALGIDEYGG

(19) United States
(12) Patent Application Publication
(10) Pub. No.: US 2024/0010704 A1
(43) Pub. Date: Jan. 11, 2024

(54) CHIMERIC SENSOR PROTEIN AND METHODS OF USE THEREOF

(71) Applicant: Universitaet Leipzig, Leipzig (DE)

(72) Inventors: Tobias LANGENHAN, Markkleeberg (DE); Nicole SCHOLZ, Leipzig (DE)

(73) Assignee: Universitaet Leipzig, Leipzig (DE)

(21) Appl. No.: 18/044,948

(22) PCT Filed: Sep. 23, 2021
(86) PCT No.: PCT/EP2021/076234

§ 371 Date: Mar. 10, 2023

(30) Foreign Application Priority Data
Sep. 25, 2020 (EP) 20198501.7

(51) Int. Cl. C07K 14/705; C12N 15/85; G01N 33/50; A01K 67/033; C07K 14/47

(52) U.S. Cl. CPC C07K 14/70596; C12N 15/8509; G01N 33/5085; A01K 67/0339; C07K 14/705; C07K 14/4702; C12N 2015/859; G01N 2333/43573; A01K 2267/01; A01K 2227/706; A01K 2267/0393; A01K 2217/05; C07K 2319/03; C07K 2319/50

(57) ABSTRACT

The present invention provides polypeptides and nucleic acid molecules that are useful in identifying.

### PRIORITY

The instant application corresponds to the U.S. National phase of International Application No. PCT/EP2021/076234, filed Sep. 23, 2021, which, in turn, claims priority to European Patent Application No. 20198501.7.3 filed Sep. 25, 2020, the contents of which are incorporated by reference herein in their entirety.

### SEQUENCE LISTING

The instant application contains a Sequence Listing that has been submitted in ASCII format via EFS-Web and is hereby incorporated by reference in its entirety. Said ASCII copy, created on Mar. 10, 2023, is named LNK_246US_SL.txt and is 147,818 bytes in size.

### FIELD OF THE PRESENT INVENTION

The present invention provides chimeric polypeptides that are useful in identifying modulators of adhesion G protein-coupled receptors and Polycystin-1/Polycystin-1-like proteins. The invention further provides screening methods using the novel polypeptides.

### BACKGROUND OF THE PRESENT INVENTION

Adhesion G protein-coupled receptors (aGPCRs) and Polycystin-1/Polycystin-1-like (PC1/PC1-like) proteins are involved in physiological cell and organ functions. Genetic aberrations and the resulting functional impairments in these molecules play a role in human diseases.¹⁻⁷ aGPCRs and PC1/PC1-like molecules form two classes of cell membrane receptors with 7 and 1/11 transmembrane segments, respectively (FIG. 1). They share structural similarities by possessing a GPCR autoproteolysis-inducing (GAIN) domain and additional adhesion domains in their extracellular region (ECR), and an intracellular region (ICR) (FIG. 2A).

The physical dissociation of aGPCRs and PC1/PC1-like molecules (FIG. 2B) is enabled by the autoproteolytic processing of the receptor proprotein generating a NTF/CTF (N-terminal fragment/C-terminal fragment) heterodimer joined at the GAIN domain⁹ (FIGS. 1 and 2). This process occurs in the endoplasmic reticulum or early Golgi apparatus upon folding of the GAIN domain.¹⁰ The GAIN domain cleaves the receptor precursor at the GPS (GPCR proteolysis site) and stabilizes the NTF-CTF heterodimer through non-covalent interactions^{9, 11-15} (FIGS. 1 and 2 A). If such forces were to overcome, for example, upon engagement of the NTF with ligands, mechanical stimulation or a combination thereof, disjunction of the heterodimer occurs through force transmission to the NTF-CTF complex (FIG. 2B) (reviewed in refs.^{3-5, 8, 16}).

NTF-CTF separation is predicted to expose a short β-sheet motif C-terminal of the GPS^{4, 17} This motif has been previously shown to harbour receptor stimulating activity by acting as a tethered agonist (TA)^{18, 19}. aGPCR activation results in the stimulation of canonical intracellular second messenger cascades and thereby cell-autonomously governs biochemical and cell biological states (reviewed in ref.⁷) Presence and principal utility of TAs has since been demonstrated for numerous aGPCRs and Polycystin-1 (reviewed in refs.^{6, 20, 36}) (FIG. 3).

Based on these observations, it appears critical to detect GAIN domain proteolysis and NTF-CTF separation as initiating steps for aGPCR and PC1/PC1-like molecule activation (FIG. 4), in order to study its occurrence, effects and possible modulation by physiological and artificial means.

To date, the family of adhesion G protein-coupled receptors (aGPCRs) consists of 33 members in humans. Many reports have demonstrated critical functions for members of this family in organogenesis, neurodevelopment, myelination, angiogenesis, and cancer progression. Importantly, mutations in several aGPCRs have been linked to human diseases. Furthermore, overexpression in cancer cells of aGPCRs has been reported. Yet, there are no approved drugs specifically targeting these proteins.

To date, there are no screening methods for identifying molecules that affect GAIN domain proteolysis and/or NTF-CTF separation, in particular in a high-throughput format.

WO 2019/099689 A1 discloses chimeric polypeptides containing a force sensor cleavage domain and methods of use thereof. WO 2019/099689 A1 defines the term “force sensor cleavage domain” as referring to a polypeptide domain of a force sensitive protein that, upon the application of force, is cleavable, e.g., by a protease. This contrasts with the GAIN domain of aGPCRs and PC1/PC1-like molecules, which is cleaved at a GPS site within the GAIN domain first and then disjoins upon force transmission acting on the NTF (N-terminal fragment), overcoming the non-covalent interactions holding together the NTF/CTF heterodimer. Furthermore, in contrast to the chimeric polypeptide defined in WO 2019/099689 A1 the aGPCR/PC1/PC1-like GAIN domain facilitates autocatalytic cleavage and hence constitutes both enzyme and substrate in one structure.

Importantly, WO 2019/099689 A1 does not disclose chimeric polypeptides comprising a GAIN domain or even mention a GAIN domain. According to WO 2019/099689 A1 one possible force sensor cleavage domain, among a list of possibilities, is an “adhesion-GPCR cleavage domain”, which may be derived from an aGPCR protein (e.g. Drosophila Flamingo protein) or a homolog thereof (i.e. CELSR1, CELSR2 and CELSR3). These aGPCR are large, e.g. about 3000 amino acid long proteins, which may comprise a GAIN domain, but also multiple other protease cleavage sites^{16, 40-46}.

WO 2019/099689 A1 does not further define “adhesion-GPCR cleavage domain” but gives with SEQ ID NO: 272-274 a number of examples for amino acid sequences of such an “adhesion-GPCR cleavage domain”. These sequences are far too short to constitute a complete GAIN domain and even if comprising part of a GAIN domain sequence they would not be expected to properly fold into a GAIN domain and would consequently not be able function as the “adhesion-GPCR cleavage domain”.

In light of all the above, there is a need for a screening method as outlined above, in particular for a method to identify inhibitors of GAIN domain proteolysis and/or NTF-CTF separation.

### SUMMARY OF THE PRESENT INVENTION

The inventors surprisingly found that GAIN domain proteolysis and/or NTF-CTF separation can be detected using a chimeric sensor protein comprising the extracellular region of an aGPCR, a Notch transmembrane domain, and an intracellular reporter moiety. The sensor protein of the present invention allows the identification of compounds that modulate the activation of aGPCR molecules or PC1/PC1-like molecules, e.g. by affecting GAIN domain proteolysis and/or NTF-CTF separation.

The present invention therefore relates to the subject matter defined in the following items [1] to [34]:

[1] A polypeptide comprising (i) a first sequence comprising a GPCR autoproteolysis-inducing (GAIN) domain of an adhesion GPCR or of a PC1/PC1-like protein, (ii) a second sequence comprising the transmembrane region of a Notch receptor, and (iii) a third sequence comprising a transcription factor moiety.
[2] The polypeptide of item [1], wherein the first sequence comprises or consists of a fragment of the extracellular region (ECR) of the aGPCR or PC1/PC1-like protein, wherein said fragment comprises said GAIN domain.
[3] The polypeptide of item [2], wherein said fragment does not comprise the N-terminal end of said aGPCR or PC1/PC1-like protein; and/or wherein said fragment does not comprise the full amino acid sequence that N-terminally precedes the GAIN domain of said aGPCR or PC1/PC1-like protein.
[4] The polypeptide of item [2] or [3], wherein said fragment has the structure X-G-Y, wherein G is the GAIN domain, X is an amino acid sequence adjacent to the N-terminal end of the GAIN domain in said aGPCR or PC1/PC1-like protein, and Y is absent or an amino acid sequence adjacent to the C-terminal end of the GAIN domain in said aGPCR or PC1/PC1-like protein.
[5] The polypeptide of item [4], wherein X consists of 1 to 7,000, or 10 to 6,000, or 100 to 5,000, or 200 to 4,000, or 300 to 3,000, or 400 to 2,000, or 500 to 1,000 amino acids.
[6] The polypeptide of item [4] or [5], wherein Y consists of 1 to 30 amino acids, or 2 to 20 amino acids, or 3 to 10 amino acids.
[7] The polypeptide of item [1], wherein the first sequence comprises or consists of the N-terminal part of the aGPCR or PC1/PC1-like protein, from the N-terminal amino acid of the aGPCR or PC1/PC1-like protein to the C-terminal end of the GAIN domain of the aGPCR or PC1/PC1-like protein.
[8] The polypeptide of item [1] or [7], wherein the first sequence comprises or consists essentially of the complete extracellular region (ECR) of said aGPCR or said PC1/PC1-like protein, such that the first sequence of the polypeptide ends before the start of the transmembrane (TM) domain of the aGPCR or the PC1/PC1-like protein.
[9] The polypeptide of any one of the preceding items, wherein the aGPCR is selected from the group of aGPCRs listed in Table 1 hereinbelow.
[10] The polypeptide of item [1], [7], [8] or [9], wherein the first sequence comprises or consists of the extracellular region of an aGPCR selected from the group of aGPCRs listed in Table 1 hereinbelow.
[11] The polypeptide of any one of items [1] to [8], wherein the PC1/PC1-like protein is selected from the group of PC1/PC1-like proteins listed in Table 2 hereinbelow.
[12] The polypeptide of any one of items [1], [7], [8] or [11], wherein the first sequence comprises or consists of the extracellular region of an PC1/PC1-like protein selected from the group of PC1/PC1-like proteins listed in Table 2 hereinbelow.
[13] The polypeptide of any one of the preceding items, wherein the GAIN domain comprises or consists of (i) an amino acid sequence selected from the group consisting of SEQ ID NOs:1-37; or (ii) an amino acid sequence having a sequence identity of at least 90% to any one of SEQ ID NOs:1-37.
[14] The polypeptide of any one of the preceding items, wherein the GAIN domain comprises or consists of an amino acid sequence selected from the group consisting of SEQ ID NOs:1-32.
[15] The polypeptide of any one of items [1] to [13], wherein the GAIN domain comprises or consists of an amino acid sequence selected from the group consisting of SEQ ID NOs:33-37.
[16] The polypeptide of any one of the preceding items, wherein the second sequence comprises the S2 cleavage site of a Notch receptor.
[17] The polypeptide of any one of the preceding items, wherein the second sequence comprises the S3 cleavage site and optionally the S4 cleavage site of the Notch receptor.
[18] The polypeptide of any one of the preceding items, wherein the second sequence comprises or consists essentially of a fragment of the Notch receptor comprising the S2 and the S3 cleavage sites, and optionally the S4 cleavage site.
[19] The polypeptide of any one of the preceding items, wherein the second sequence consists essentially of the iuxtamembrane segment and the transmembrane region of the Notch receptor.
[20] The polypeptide of any one of the preceding items, wherein the second sequence comprises or consists essentially of (i) an amino acid sequence selected from the group consisting of SEQ ID NOs:52-56, (ii) an amino acid sequence having a sequence identity of at least 90% to any one of SEQ ID NOs:52-56, (iii) an amino acid sequence which differs from any one of SEQ ID NOs:52-56 in less than 10, or less than 7, or less than 5, or less than 3 amino acids, e.g. in one or two amino acids, or (iv) a fragment of (i), (ii) or (iii) comprising the S2 and the S3 cleavage sites.
[21] The polypeptide of any one of the preceding items, wherein the transcription factor moiety is selected from the group consisting of LexA from E. coli, GAL4 from S. cerevisiae, QF, QF2 and QF2w derived from N. crassa.
[22] The polypeptide of any one of the preceding items, wherein the transcription factor moiety, upon separation from the second sequence, is capable of activating transcription of a reporter gene.
[23] The polypeptide of any one of the preceding items, wherein release of an N-terminal portion of the first sequence induces cleavage of the second sequence at one or more proteolytic cleavage sites, thereby releasing the transcription factor moiety.
[24] The polypeptide of any one of the preceding claims, which is a sensor protein suitable for detecting the release of its N-terminal fragment and/or proteolytic cleavage within the GAIN domain.
[25] The polypeptide of item [1], having or consisting essentially of the amino acid sequence as shown in SEQ ID NO:59.
[26] The polypeptide of any one of the preceding items, which is embedded in a biological membrane, e.g. in a cell membrane.
[27] The polypeptide of any one of the preceding items, which is integrated in the membrane of a cell, wherein the first sequence extends to the extracellular/luminal space, the transmembrane region is located within the membrane, and the transcription factor moiety extends to the intracellular/cytoplasmic space.
[28] A nucleic acid encoding the polypeptide of any one of the preceding items.
[29] A plasmid or vector comprising the nucleic acid of item [28].
[30] A cell comprising the polypeptide of any one of items [1] to [27], the nucleic acid of item [28], or the plasmid or vector of item [29].
[31] The cell of item [30], further comprising a nucleic acid capable of binding to the transcription factor moiety, operably linked to a nucleic acid encoding a reporter.
[32] A non-human transgenic animal comprising the polypeptide of any one of items [1] to [27], the nucleic acid of item [28], the plasmid or vector of item [29], or the cell of item [30] or [31].
[33] A non-human transgenic animal expressing the polypeptide of any one of items [1] to [27].
[34] A screening method comprising the following steps
(a) contacting a test compound with the cell of item [30] or [31], or administering a test compound to the transgenic animal of item [32] or [33] and
(b) determining the level of the reporter or of a detectable signal caused or generated by the reporter.
   [35] A method for identifying modulators of an aGPCR or of a PC1/PC1-like protein comprising the following steps
(a) contacting a test compound with the cell of item [30] or [31], or administering a test compound to the transgenic animal of item [32] or [33] and
(b) determining the level of the reporter or of a detectable signal caused or generated by the reporter.
   [36] The method of item [34] or [35], wherein the modulator is an inhibitor.
   [37] The method of any one of items [34] to [36], wherein the cell or the transgenic animal comprises (i) a nucleic acid sequence which is capable of binding to the transcription factor moiety of the polypeptide upon release of the transcription factor moiety from the polypeptide, and (ii) a nucleic acid sequence encoding a reporter; wherein expression of the reporter is induced when the nucleic acid sequence which is capable of binding to the transcription factor moiety binds to the transcription factor moiety.
   [38] The method of any one of items [34] to [37], further comprising the steps of comparing the level determined in step (b) with a control level, and selecting the test compound if the level determined in step (b) is lower than the control level.
   [39] The use of the polypeptide of any one of items [1] to [27], the nucleic acid of item [28], the plasmid or vector of item [29], the cell of item [30] or [31], or of the transgenic of item [32] or [33] for identifying a modulator of an adhesion GPCR or of a PC1/PC1-like protein, preferably an inhibitor of an adhesion GPCR or of a PC1/PC1-like protein.

### BRIEF DESCRIPTION OF THE FIGURES

FIG. 1. Structural similarities between adhesion GPCRs and PC1/PC1-like proteins.

FIGS. 2A-2B. FIG. 2A depicts adhesion GPCRs and PC1/PC1-like proteins that are composed of a large extracellular region (ECR), heptahelical transmembrane (7TM) domain, and an intracellular region (ICR). FIG. 2B depicts the GAIN domain that is autoproteolytically active and generates an N- (NTF) and C-terminal fragment (CTF) from each adhesion GPCR/PC1/PC1-like preprotein. The two fragments form a non-covalently stabilised heterodimer through non-covalent interactions.

FIG. 3. Amino acid alignment of the protein region at the GPS of adhesion GPCRs and PC1/PC1-like proteins highlighting the high evolutionary conservation of cleavage site and tethered agonist segment of both protein classes (SEQ ID NOs:38-51).

FIG. 4. The dissociation model proposes that aGPCR activation involves separation of the NTF/CTF heterodimer and NTF release.

FIG. 5. Molecular design of a polypeptide of the invention, also referred to herein as NTF Release Sensor (NRS) protein. ECR, extracellular region; GPS, GPCR proteolysis site; ITS, iuxta- and transmembrane segment; TA, tethered agonist; TF, transcription factor subunit.

FIG. 6. Functionality of the NRS is based on the regulated intramembrane proteolysis of Notch, and couples NTF release to the intracellular release and intranuclear activity of heterologous transcription factors (TF).

FIGS. 7A-7B. FIG. 7A demonstrates that NRS signaling is repressable by fusion with heterologous extracellular domains such as Ig domains of CD4, and can be NRS re-activated by extracellular TEVp expression. CD4-NRS-LexA vs. N^{ECN}-LexA: 37.7×; n=3, p=<0.0001; CD4-3TEVs-NRS-LexA vs. +secTEVp: 13.3×, n=3, p=0.0008; CD4-3TEVs-NRS-LexA vs. +intraTEVp: 1.2×, n=3, p=0.0335; CD4-6TEVs-NRS-LexA vs. +secTEVp: 28.6×, n=3, p=0.0004; CD4-6TEVs-NRS-LexA vs. +intraTEVp: 5.6×, n=3, p=0.0005. Unpaired t-test, two-tailed.

FIG. 7B depicts the results of a representative S2 cell reporter assay of different CD4-NRS-LexA versions. Only N^{ECN}-LexA and CD4-6TEVs-NRS-LexA activated by extracellular secTEVp results in proteolytic processing NRS and reporter gene activation. Scale bar=100 μm.

FIGS. 8A-8C. FIG. 8A depicts the alignment of the ITS region of Drosophila Notch (SEQ ID NO:57) and mouse Notch (SEQ ID NO:58), positions of S2, S3 and S4 sites are indicated²⁴. Predicted transmembrane helix boxed in light grey, S3 site Valine residue mutated in NRS constructs in dark grey.

FIG. 8B depicts γ-secretase substrate recognition by a critical S3 site mutation that abrogates NRS responses. Representative luciferase activity assay. N^{ECN}-LexA vs. N^{ECN/S3}-LexA: 7.9×, n=4, p=0.0009; CD4-6TEVs-NRS-LexA vs. +secTEVp: 31.0×, n=4, p=0.0005; CD4-6TEVs-NRS^{S3}-LexA vs. +secTEVp: 1.6×, n=4, p=0.0091. Unpaired t-test, two-tailed.

FIG. 8C depicts the pharmacological inhibition of γ-secretase activity that suppresses NRS responses. Representative luciferase activity assay showing reporter activity without DAPT vs. with 10 μM DAPT: Control: 1.1×, n=3, p=0.8298; N^{EGF}-LexA: 5.8×, n=3, p=0.0022; N^{ECN}-LexA: 43.7×, n=3, p=0.0018; CD4-6TEVs-NRS-LexA: 3.6×, n=3, p=0.0053; CD4-6TEVs-NRS-LexA+secTEVp: 23.4×, n=3, p=0.0039; CD4-6TEVs-NRS-LexA+intraTEVp: 7.0×, n=3, p=0.0493. Unpaired t-test, two-tailed.

FIG. 9. CIRL-NRS-LexA shows constitutive activity through NTF/CTF dissociation. Representative luciferase activity assay which is quenched by preclusion of either GAIN domain autoproteolysis or S3 proteolysis through respective cleavage site mutations. N^{EGF}-LexA vs. N^{ECN}-LexA: 254.6×, n=4, p=0.0006; Cirl-NRS-LexA vs. Cirl^{H>A}-NRS-LexA: 148.8×, n=4, p=<0.0001; Cirl-NRS-LexA vs. Cirl-NRS^{S3}-LexA: 23.7×, n=4, p=<0.0001.

FIG. 10A-10C. NRS working principle tested in vivo using Drosophila melanogaster. Biochemical confirmation. FIG. 10A depicts the layout of genomically engineered cirl alleles in Drosophila melanogaster that encode various cirl-NTF-TFx transgenes. FIG. 10B depicts the layout of the gene product encoded by the cirl-NRS-LexA allele, and molecular weights of posttranslational cleavage fragments of the gene product resulting from GAIN domain, S2, S3 and S4 proteolyses. GPS, GPCR proteolysis site; FL, full-length protein; NTF, N-terminal fragment. FIG. 10C depicts the results of Western blot analysis of protein harvested from animals carrying genomically integrated cirl-NRS-LexA sensor variants. The C-terminal sensor protein portions of the wildtype (WT) sensor (black arrowhead) derives from the GPS>S2>S3 cascade. In contrast, when GAIN domain cleavage is blocked by the H>A GPS mutation, the upward band shift corresponds to the full-length sensor protein (white arrowhead). Abrogating sensor proteolysis at the S3 site of the Notch ITS fragment results in a double band indicating incomplete S3 cleavage (double arrowhead).

FIGS. 11A-11C. NRS working principle tested in vivo using Drosophila melanogaster. Anatomical confirmation through expression analysis of the CIRL-NRS-LexA sensor through induction of a transcriptionally activated mCherry chromophore. In FIG. 11A, CIRL-NRS-LexA expression was observed in proboscis (chevron), eyes (double chevron) and leg joints (boxe and inset). In FIG. 11B, this is abrogated upon mutation the S3 site within the Notch^{ITS} component of the sensor protein. In FIG. 11C, mCherry expression is also largely suppressed in flies carrying a sensor variant lacking GAIN domain proteolysis through mutation of a −2 His to Ala, a classical mutation disrupting adhesion GPCR and PC1/PC1-like protein GAIN domain proteolysis. White arrowheads, femorotibial joint; black arrowhead, tibiotarsal joint.

FIGS. 12A-12C. Expression of genomically integrated cirl-NRS-TFx sensors with different TF cassettes shows comparable expression patterns in adult animals with strong activity in the eye, proboscis and leg joints (arrowheads) of (FIG. 12A) cirl-NTF-LexA and (FIG. 12B) cirl-NTF-Ga/4. (FIG. 12C) cirl-NTF-QF2 displayed pronounced activity only in the eye. Leg joint expression was detectable at fainter levels (arrowheads).

FIG. 13. Schematic workflow of the NRS-methodology for in-vitro high throughput screening and/or compound testing in transgenic animal models.

### DETAILED DESCRIPTION OF THE PREFERRED EMBODIMENTS

The present invention relates to a polypeptide comprising (i) a first sequence comprising a GAIN domain of an adhesion GPCR or of a PC1/PC1-like protein (ii) a second sequence comprising the transmembrane region of a Notch receptor, and (iii) a third sequence comprising a transcription activator.

The polypeptide of the invention is a chimeric transmembrane protein comprising a single-pass transmembrane domain.

### First Sequence

The term “adhesion GPCR” or “aGPCR” refers to a G-protein coupled receptor protein of class B subfamily 2³⁷ characterized by a 7 transmembrane domain and an extracellular region (ECR) with a GAIN domain. It does not necessarily need to but can additionally contain one or more adhesion domains within its ECR.

The term “adhesion domain” refers to autonomously folding protein subunits that permit interactions with ligands contained within the extracellular matrix, mounted on the same (cis) or neighbouring (trans) cell surfaces. Adhesion domains mediate but are not limited to one or more of the following functions: adhesive cell anchoring, formation of cell junctions, cell migration, cell polarisation, tissue polarisation, interaction with fixed or soluble ligands, transmission of mechanical signals onto expressing cells.

The terms “GPCR autoproteolysis-inducing domain” and “GAIN domain”, as used herein, refer to a region within the ECR of an aGPCR or PC1/PC1-like protein composed of an N-terminal subdomain A and a C-terminal subdomain B (see refs. 9, 38 and 39). Subdomain A of the GAIN domain is composed of 3 to 6 α-helices. Subdomain B of the GAIN domain is composed of 13 β-strands and can additionally contain several small α-helices. Subdomain B typically forms a twisted β-sandwich structure and contains the GPCR Proteolysis Site (GPS). Based on primary protein sequences these properties can be predicted by publicly available software tools such as Phyre2 (http://www.sbg.bio.ic.ac.uk/˜phyre2), Psipred (http://bioinf.cs.ucl.ac.uk/psipred/) and DALI (http://ekhidna2.biocenter.helsinki.fi/dali/). Primary sequence identity between orthologous GAIN domains can be as low as 10%, but the Root Mean Square Deviation (RMSD) of atomic positions of the folded GAIN domain is <5 Å.

For example, the GAIN domain of rat CL1 (UniProtKB—O88917/AGRL1_RAT) extends from Thr533-Ile849 of the amino acid sequence of the receptor. Using structure-based searches such as DALI and BLAST one can determine for other aGPCRs or PC1/PC1-like proteins which amino acids constitute the GAIN domain. For example, the GAIN domains of several human aGPCRs are shown in SEQ ID NOs:1-32 (see also Table 1 below), and the GAIN domains of several human PC1/PC1-like proteins are shown in SEQ ID NOs:33-37 (see also Table 2 below). The GAIN domain may or may not induce autoproteolysis in the polypeptide of the invention. The term “GAIN” domain includes domains which do not exert autoproteolytic activity, but are classified as GAIN domains on the basis of alignments and/or sequence similarity to other proteins known to have a GAIN domain. Some adhesion GPCRs and PC1 molecules contain autoproteolytic domains in addition to the GAIN domain, some are processed by other endogenous proteases. All three modes of cleavage (through the GAIN domain, through non-GAIN domains of the receptors, through endogenous proteases) can be detected using the polypeptide of the invention and are encompassed by the present invention.

The aGPCRs in the sense of the present invention include, but are not limited to, human aGPCRs and non-human orthologs thereof (e.g. from mammalians such as mouse, rat, Macaca mulatta or Macaca fascicularis, other vertebrates such as Xenopus tropicalis and Danio rerio, and invertebrates such as Drosophila melanogaster or Caenorhabditis elegans). Preferably, the aGPCR is a human aGPCR. More preferably, the aGPCR is selected from the aGPCRs listed in Table 1.

TABLE 1
Human aGPCRs.

| | | Human | | GAIN domain |
|---|---|---|---|---|
| | | nucleotide | Human | sequence |
| Name | Synonym(s) | RefSeq | SwissProt | (SEQ ID NO) |
| ADGRA2 | GPR124 | NM_032777 | Q96PE1 | 1 |
| ADGRA3 | GPR125 | NM_145290 | Q8IWK6 | 2 |
| ADGRB1 | BAI1 | NM_001702 | O14514 | 3 |
| ADGRB2 | BAI2 | NM_001294335 | O60241 | 4 |
| ADGRB3 | BAI3 | NM_001704 | O60242 | 5 |
| ADGRC1 | CELSR1 | NM_014246 | Q9NYQ6 | 6 |
| ADGRC2 | CELSR2 | NM_001408 | Q9HCU4 | 7 |
| ADGRC3 | CELSR3 | NM_001407 | Q9NYQ7 | 8 |
| ADGRD1 | GPR133 | NM 198827 | Q6QNK2 | 9 |
| ADGRD2 | GPR144 | | Q7Z7M1 | 10 |
| ADGRE1 | EMR1 | NM_001974; | Q14246 | 11 |
| | | NM_001256252; | | |
| | | NM_001256253; | | |
| | | NM_001256254; | | |
| | | NM_001256255 | | |
| ADGRE2 | EMR2; CD312 | NM_013447 | Q9UHX3 | 12 |
| ADGRE3 | EMR3 | NM_032571 | Q9BY15 | 13 |
| ADGRE4P | EMR4; GPR127 | NR_024075 | Q86SQ3 | 14 |
| ADGRE5 | CD97 | NM_078481; | P48960 | 15 |
| | | NM_001025160; | | |
| | | NM_001784 | | |
| ADGRF1 | GPR110 | NM_153840 | Q5T601 | 16 |
| ADGRF2 | GPR111 | NM_153839 | Q8IZF7 | 17 |
| ADGRF3 | GPR113 | NM_153835 | Q8IZF5 | 18 |
| ADGRF4 | GPR115 | NM_153838 | Q8IZF3 | 19 |
| ADGRF5 | GPR116 | NM_015234 | Q8IZF2 | 20 |
| ADGRG1 | GPR56 | NM_005682 | Q9Y653 | 21 |
| ADGRG2 | GPR64 | NM_001079858; | Q8IZP9 | 22 |
| | | NM_001079859; | | |
| | | NM_001079860; | | |
| | | NM_005756; | | |
| | | NM_001184833; | | |
| | | NM_001184834; | | |
| | | NM_001184835; | | |
| | | NM_001184836 | | |
| ADGRG3 | GPR97 | NM_170776 | Q86Y34 | 23 |
| ADGRG4 | GPR112 | NM_153834 | Q8IZF6 | 24 |
| ADGRG5 | GPR114 | NM_153837 | Q8IZF4 | 25 |
| ADGRG6 | GPR126 | NM_020455; | Q86SQ4 | 26 |
| | | NM_001032394; | | |
| | | NM_001032395; | | |
| | | NM_198569 | | |
| ADGRG7 | GPR128 | NM_032787 | Q96K78 | 27 |
| ADGRL1 | LPHN1; CIRL1 | NM_014921; | O94910 | 28 |
| | | NM_001008701 | | |
| ADGRL2 | LPHN2; CIRL2 | NM_012302 | O95490 | 29 |
| ADGRL3 | LPHN3; CIRL3 | NM_015236 | Q9HAR2 | 30 |
| ADGRL4 | ELTD1 | NM_022159 | Q9HBW9 | 31 |
| ADGRV1 | VLGR1; GPR98 | NM_032119 | Q8WXG9 | 32 |

The term “PC1/PC1-like protein” as used herein refers to polypeptides comprising a 1 or 11 transmembrane domain and an extracellular region with a GAIN domain, PKD domains and adhesion domains. Preferably the PC1/PC1-like protein in accordance with this invention is a human PC1/PC1-like protein. In another preferred embodiment the PC1/PC1-like protein is selected from the group consisting of human Polycystin-1 (hPC1; also known as hPKD1), hPC1L1 (also known as hPKDL1), hPC1L2 (also known as hPKDL2), hPC1L3 (also known as hPKDL3), hPKDREJ and non-human orthologs thereof (e.g. from mammalians such as mouse, rat, Macaca mulatta or Macaca fascicularis).

TABLE 2
Human PC1/PC1-like proteins.

| | | Human | | GAIN domain |
|---|---|---|---|---|
| | | nucleotide | Human | sequence |
| Name | Synonym(s) | RefSeq | SwissProt | (SEQ ID NO) |
| PC1 | PKD1 | NM_000296.3; | P98161 | 33 |
| | | NM_001009944.2 | | |
| PC1L1 | PKDL1 | NM_138295.4 | Q8TDX9 | 34 |
| PC1L2 | PKDL2 | NM_001076780.1; | Q7Z442 | 35 |
| | | NM_001278423.1; | | |
| | | NM_001278425.1; | | |
| | | NM_052892.3 | | |
| PC1L3 | PKDL3 | NM_181536.1 | Q7Z443 | 36 |
| PKDREJ | | NM_006071 | Q9NTG1 | 37 |

The full length of the polypeptide of the invention may range from about 200 to about 10,000 amino acids, preferably from about 500 to about 8,000 amino acids, or from about 1,000 to about 6,000 amino acids.

The first sequence of the polypeptide of the invention typically has a length from about 150 to about 7,000 amino acids, preferably from about 200 to about 6,000 amino acids, or from about 300 to about 5,000 amino acids, or from 400 to 4,000 amino acids. The first sequence may comprise at least two adhesion domains, preferably at least three or at least four or at least five adhesion domains.

In one embodiment the first sequence comprises, or consists of, a fragment of the extracellular region (ECR) of the aGPCR or PC1/PC1-like protein, wherein said fragment comprises said GAIN domain. In certain embodiments the fragment does not comprise the full amino acid sequence that N-terminally precedes the GAIN domain of said aGPCR or PC1/PC1-like protein. In other embodiments the fragment does not comprise the N-terminal end of said aGPCR or PC1/PC1-like protein. In another embodiment, the first sequence comprises or consists of the N-terminal amino acid sequence of an aGPCR extending from the N-terminus of the aGPCR to the C-terminus of the GAIN domain. In another embodiment the first sequence comprises or consists essentially of the complete ECR of the aGPCR, i.e. the first sequence of the polypeptide ends before the start of the TM domain of the aGPCR. In yet other embodiments the first sequence comprises or consists essentially of the N-terminal amino acid sequence of a PC1/PC1-like protein from the N-terminus to the C-terminus of the GAIN domain. In another embodiment the first sequence comprises or consists essentially of the complete ECR of the PC1/PC1-like protein, i.e. the first sequence of the polypeptide ends before the start of the TM domain of the PC1/PC1-like protein.

In a specific embodiment, the first sequence comprises or consists essentially of (a) an amino acid sequence selected from the group consisting of SEQ ID NOs:1-37, (b) an amino acid which has a sequence identity of at least 90% to any one of SEQ ID NOs:1-37, or (c) an amino acid sequence which differs from an amino acid sequence as shown in any one of SEQ ID NOs:1-37 in less than 50, or less than 25, or less than 10, or less than 5, or less than 3 amino acids.

In another embodiment, the first sequence comprises or consists essentially of (a) an amino acid sequence selected from the group consisting of SEQ ID NOs:1-32, (b) an amino acid which has a sequence identity of at least 90% to any one of SEQ ID NOs:1-32, or (c) an amino acid sequence which differs from an amino acid sequence as shown in any one of SEQ ID NOs:1-32 in less than 50, or less than 25, or less than 10, or less than 5, or less than 3 amino acids.

In yet another embodiment, the first sequence comprises or consists essentially of (a) an amino acid sequence selected from the group consisting of SEQ ID NOs:33-37, (b) an amino acid which has a sequence identity of at least 90% to any one of SEQ ID NOs:33-37, or (c) an amino acid sequence which differs from an amino acid sequence as shown in any one of SEQ ID NOs:33-37 in less than 50, or less than 25, or less than 10, or less than 5, or less than 3 amino acids.

The difference in amino acid sequence may be a substitution, deletion and/or insertion relative to the reference sequence. The comparison of sequences and determination of percent identity between two amino acid sequences can be accomplished using any suitable program, e.g. the program “BLAST 2 SEQUENCES (blastp)” (Tatusova et al. (1999) FEMS Microbiol. Lett. 174, 247-250) with the following parameters: Matrix BLOSUM62; Open gap 11 and extension gap 1 penalties; gap x_dropoff50; expect 10.0 word size 3; Filter: none.

In another preferred embodiment the first sequence comprises one of the amino acid sequences shown in FIG. 3 (SEQ ID NOs:38-51).

In other embodiments, the first sequence comprises or consists of the GAIN domain of one of the following aGPCRs:

mGPR133, rGPR128, rGPR126, rGPR116, mGPR110, rGPR64, mCELSR3, rCELS, mGPR125, mGPR124, rCD97, rEMR1, mEMR4, mGPR97, rGPR114, rETL1, mGPR115, mGPR111, mVLGR1, rGPR113, rBA11, rBAI2, and rCL1. These GAIN domain sequences are disclosed in Supplementary Table 3 of Arac et al. EMBO J 31, 1364-1378 (2012), which sequences are incorporated herein by reference.

In yet other embodiments, the first sequence comprises or consists of the GAIN domain of one of the following aGPCRs: TT89292280, TT89298346, DM48958429, TT89296699, CE115532418, MB16753744x7, MB167524088, MB238058440, TA195996857, DD66815909, TA196016767, CE35396740, TA196004662, and DM19527793. These GAIN domain sequences are disclosed in Supplementary Table 2 of Arac et al. EMBO J31, 1364-1378 (2012), which sequences are incorporated herein by reference.

The GAIN domain in accordance with this invention includes splice variants, isoforms, human single nucleotide polymorphisms (SNPs) and pathophysiologically relevant human mutations of any one of the GAIN domains mentioned above.

### Second Sequence

The second sequence comprises the transmembrane region of a Notch receptor. The Notch receptor can be a human Notch receptor. In one embodiment the Notch receptor is selected from the group consisting of Notch1, Notch2, Notch3, and Notch4. The amino acid sequence of human Notch1 can be found in UniProtKB—P46531. The amino acid sequence of human Notch2 is described in UniProtKB—004721. The amino acid sequence of human Notch3 is described in UniProtKB—Q9UM47. The amino acid sequence of human Notch4 is described in UniProtKB—Q99466. In other embodiments the Notch receptor is a mammalian orthologue of one of the human Notch proteins mention above.

The length of the second sequence may range from about 40 amino acids to about 100 amino acids, preferably from about 45 to about 75 amino acids, or from about 50 to about 60 amino acids.

The second sequence typically includes the S2 cleavage site which is located in the extracellular part of the Notch protein, adjacent to the TM domain. The S2 cleavage site can be cleaved by the matrix metalloprotease of the Kuzbanian/ADAM (a disintegrin and metalloprotease domain) family. The short segment from the amino acids constituting the S2 cleavage site of a Notch receptor to the C-terminal end of the extracellular region of the Notch receptor is referred to as iuxtamembrane segment herein. For the avoidance of doubt, the iuxtamembrane segment includes the S2 cleavage site, but it does not include amino acids of the transmembrane region of the Notch receptor. Thus, the second sequence typically comprises or consists essentially of the iuxtamembrane segment and the transmembrane region of a Notch receptor. For example, the iuxtamembrane segment of human Notch 1 includes amino acids 25-42 of SEQ ID NO:52. The structure and function of Notch receptors is reviewed in ref.²⁴

The second sequence usually further comprises the S3 cleavage site, which is located within the TM portion of Notch. In yet another embodiment the second sequence further comprises the S4 cleavage site, which is located within the TM portion of Notch.

In a preferred embodiment the second sequence comprises or consists essentially of (i) an amino acid sequence selected from the group consisting of SEQ ID NOs:52-56, (ii) an amino acid sequence having a sequence identity of at least 90% to any one of SEQ ID NOs:52-56, (iii) an amino acid sequence which differs from any one of SEQ ID NOs:52-56 in less than 10, or less than 7, or less than 5, or less than 3 amino acids, e.g. in one or two amino acids, or (iv) a fragment of (i), (ii) or (iii) comprising the S2 and the S3 cleavage sites. The fragment may have an N-terminal truncation of any one of SEQ ID NOs:52-56, by 1-25 amino acids, by 1-24 amino acids, by 1-23 amino acids, by 1-22 amino acids, by 1-21 amino acids, or by 1-20 amino acids. The fragment may have a C-terminal truncation of any one of SEQ ID NOs:52-56, by 1, 2, 3, 4 or 5 amino acids. The embodiments concerning the N-terminal truncation and the C-terminal truncation can be combined.

### Third Sequence

The polypeptide of the invention further comprises, at the C-terminal end, a transcription factor moiety.

The length of the third sequence may range from about 50 amino acids to about 1,500 amino acids, or from about 100 to about 1,000 amino acids.

Upon release from the second sequence, e.g. by proteolytic cleavage, the transcription factor moiety can bind to a DNA target sequence, thereby inducing transcription of a reporter gene. The type of the reporter gene is not particularly limited, provided that it encodes an expression product that can be detected, or which provides a detectable signal. Suitable transcription factors include, but are not limited to, LexA from Escherichia coli²¹, GAL4 from Saccharomyces cerevisiae²², and QF, QF2 and QF2^{w} derived from Neurospora crassa²³, for which corresponding transcription factor DNA binding motifs for the construction of reporter transgenes are known.

Optionally, there can be a spacer between the first sequence and the second sequence, and/or between the second and the third sequence. The spacer does not affect the functionality of the chimeric polypeptide. The length of the spacer may range from about 1 to about 10 amino acids. The sequence of the spacer may be heterologous to the adjacent sequences. Preferably, no spacer is present between the first sequence and the second sequence. In another embodiment, no spacer is present between the second and the third sequence. In yet another embodiment, no spacer is present between the first sequence and the second sequence, nor between the second and the third sequence. In a specific embodiment, no spacer is present between the first sequence and the second sequence, and a spacer is present between the second and the third sequence.

In other optional embodiments, the polypeptide of the invention comprises at its N-terminal end an amino acid sequence that is heterologous to the aGPCR sequence or PC1/PC1-like sequence that is present in the first sequence. Examples of such heterologous sequences include tag sequences, signal peptides and the like.

In other optional embodiments, the polypeptide of the invention comprises at its C-terminal end an amino acid sequence that is heterologous to the transcription factor sequence of that is present in the third sequence. Examples of such heterologous sequences include tag sequences and the like.

### Nucleic Acids

The present invention further relates to a nucleic acid encoding the polypeptide of the invention. The nucleic acid sequences encoding various aGPCRs and Notch proteins are known. Similarly, the nucleic acid sequences encoding various transcription factor moieties are known. Based on the teaching of the present application the skilled person is able to design and prepare suitable nucleic acid constructs encoding the polypeptide described herein.

The invention further pertains to a plasmid or vector comprising a nucleic acid of the invention. The vector is preferably an expression vector. Typical expression vectors contain promoters that direct the synthesis of large amounts of mRNA corresponding to the inserted nucleic acid in the plasmid-bearing cells. They may also include an origin of replication sequence allowing for their autonomous replication within the host organism, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

### Cells

The present invention further relates to a cell comprising the polypeptide of the present invention. The invention further pertains to a cell comprising the nucleic acid of the present invention. The invention further pertains to a cell comprising the plasmid or vector of the present invention. The invention further pertains to a cell expressing the polypeptide of the present invention. The cell of the invention is obtainable by a process comprising the steps of introducing a nucleic acid encoding the polypeptide of the invention into a host cell, and culturing the cells so obtained under conditions that allow expression of the polypeptide.

Any cell can be chosen as long as the polypeptide as expressed is functional. In one embodiment, the expression of the polypeptide is stable, optionally inducible. Alternatively, the expression of the polypeptide is transient. Preferably, the cell is a eukaryotic cell. More preferably, the cell is an insect or a mammalian cell. Even more preferably, the mammalian cell is a human cell. Examples of mammalian cells are HEK293, HEK293T, MDCK, CHO, COS, NIH3T3, Swiss3T3, BHK, and A549. Even more preferably, the cell is a mammalian cell such as HEK293.

Depending on the type of expression system chosen, the skilled person may possibly adapt the culture conditions to obtain a most favorable expression level of the polypeptide. In the case of an inducible expression system, the skilled person may also possibly optimize an inducing condition. The time period of induction of the expression and the temperature during induction of the expression could also possibly be optimized.

Typically, the cells to be provided are obtained by introducing the nucleic acid encoding a polypeptide of the invention into host cells, e.g. mammalian host cells. Typically, the host cell is transfected with a suitable plasmid or nucleic acid, or transduced with a suitable vector, e.g. a viral vector.

The cell of the invention preferably includes a heterologous nucleic acid sequence which is capable of binding to the transcription factor moiety comprised in the third sequence. Suitable transcription factor binding DNA sequences include, but are not limited to, QUAS (for binding to transcription activators of the Q system, such as QF or QF2), UAS (for binding to transcription activators such as Gal4) and LexAop (for binding to transcription activators such as LexA).

It is further preferred that the cell of the invention comprises a heterologous nucleic acid sequence encoding a reporter. Suitable reporters include, e.g., fluorescent proteins; enzymes that catalyze a reaction that generates a detectable signal as a product, and the like.

Suitable fluorescent proteins are described in Shaner et al. (2005) Nat. Methods 2:905-909) and Matz et al. (1999) Nature Biotechnol. 17:969-973, the content of which is incorporated herein.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, Xanthine Oxidase, firefly luciferase, glucose oxidase (GO), and the like.

Preferred reporters are firefly luciferase, green fluorescent protein and red fluorescent proteins such as mCherry. The nucleic acid sequences encoding reporters are known, as well as suitable promoters to be operably linked to the nucleic acid encoding the reporter.

Such cells are obtained by additionally introducing a nucleic acid encoding the reporter into the cell.

### Transgenic Animals

The invention further pertains to a non-human transgenic animal comprising the polypeptide of the invention, the nucleic acid of the invention, the plasmid or vector of the invention, or the cell of the invention. The invention further relates to a non-human transgenic animal expressing the polypeptide of the present invention.

The transgenic non-human animal may be a transgenic non-human vertebrate animal, preferably a mammal, preferably a rodent, such as a mouse. Suitable animals are available, or easily generated, using conventional methods, in a variety of genera, including rodents (e.g., rats), rabbits, guinea pigs, dogs, goats, sheep, cows, horses, pigs, llamas, camels or the like. Preferably, the non-human transgenic animal is a transgenic mouse.

The invention further provides a transgenic gamete, including a transgenic ovum or sperm cell, a transgenic embryo, and any other type of transgenic cell or cluster of cells, whether haploid, diploid, or of higher zygosity comprising a nucleic acid of the present invention.

As used herein, the term “embryo” includes a fertilized ovum or egg (i.e., a zygote) as well as later multicellular developmental stages of the organism.

Also included herein are progeny of the transgenic animal that preferably comprises a nucleic acid of the invention.

The transgenic animal may be sterile although, preferably, it is fertile. The present invention further includes a cell line derived from a transgenic embryo or other transgenic cell of the invention, which comprises the nucleic acid of the invention and/or expresses the polypeptide of the invention. Methods of isolating such cells and propagating them are known to those of skill in the art.

Techniques for the generation of non-human transgenic animals are generally known to the skilled person (see, e.g. Pinkert, Transgenic Animal Technology: A Laboratory Handbook, ISBN-13: 978-0124104907). The transgenic non-human animals of the invention can be produced by introducing recombinant nucleic acids into the germline of the non-human animal. Embryonal target cells at various developmental stages are used to introduce the nucleic acids or vectors of the invention. Different methods are used depending on the stage of development of the embryonal target cell(s). Such methods include, but are not limited to, microinjection of zygotes, viral integration, and transformation of embryonic stem cells.

### Methods and Uses

The invention further relates to methods for identifying compound that are capable of modulating aGPCR activity. The method may be a screening method. The method typically comprises the following steps
(a) contacting a test compound with a cell expressing the polypeptide of the present invention, or administering the test compound to a transgenic animal comprising such a cell, wherein said cell comprises (i) a nucleic acid sequence which is capable of binding to the transcription factor moiety of the polypeptide upon release of the transcription factor moiety from the polypeptide, and (ii) a nucleic acid sequence encoding a reporter; wherein expression of the reporter is induced when the nucleic acid sequence which is capable of binding to the transcription factor moiety binds to the transcription factor moiety; and
(b) determining the level of the reporter or of a detectable signal caused by the reporter.

“Determining the level” can be a quantitative determination or a qualitative determination (e.g. determining whether or not a reporter or signal can be detected).

The method may further comprise one or more of the following steps
Prior to step (a), providing a cell as described above.
After step (a), culturing the cell in the presence of the test compound for at least 1 minute, or at least 1 hour, or at least 24 hours; e.g. for a time period from 1 minute to 1 week, or from 10 minutes to 24 hours, or from 1 hour to 12 hours.
Contacting the cell with a ligand that is capable of binding to the extracellular region of the polypeptide of the invention; the ligand may be added to the cells prior to the test compound, or simultaneously with the test compound; the ligand may be a ligand activating the aGPCR (or PC1/PC1-like protein) or a ligand inhibiting the aGPCR (or PC1/PC1-like protein). In one embodiment the polypeptide is constitutively active, and no ligand is added;
Comparing the level determined in step (b) with a control level, e.g. with the level in the absence of the test compound. When a ligand of the polypeptide is present when contacting the test compound with the cell the control level is usually determined in the presence of the ligand and in the absence of the test compound. When no ligand of the polypeptide is present when contacting the test compound with the cell (e.g. if the polypeptide has constitutive activity) the control level is usually determined in the absence of a ligand of the polypeptide and in the absence of the test compound.
Selecting the test compound if the level determined in step (b) is higher than the control level (when screening for activating test compounds); or selecting the test compound if the level determined in step (b) is lower than the control level (when screening for inhibiting compounds). Screening for inhibitors is preferred.

The above embodiments described for a cellular assay can be applied to methods using non-human transgenic animals mutatis mutandis.

The invention further pertains to the use of the polypeptide of the invention, of the nucleic acid of the invention, of the plasmid or vector of the invention, or of the cell of the invention, or of the transgenic animal of the invention for identifying a modulator, preferably an inhibitor, of an adhesion GPCR or of a PC1/PC1-like protein.

Modulators of an adhesion GPCR or of a PC1/PC1-like protein can be activators or inhibitors. Modulators further include agonists and antagonists. Preferably, the modulator is an inhibitor of an adhesion GPCR or of a PC1/PC1-like protein.

The invention further pertains to the use of the polypeptide of the invention for screening for agonistic ligands of an adhesion GPCR or of a PC1/PC1-like protein. In another embodiment the invention pertains to the use of the polypeptide of the invention for screening for antagonistic ligands of an adhesion GPCR or of a PC1/PC1-like protein. In another embodiment the invention pertains to the use of the polypeptide of the invention for screening for agonistic modulators of an adhesion GPCR or of a PC1/PC1-like protein. In another embodiment the invention pertains to the use of the polypeptide of the invention for screening for modulators of non-GAIN domain proteolysis of the ECR. The N-terminal fragments cleaved off from the polypeptide of the present invention can be isolated and/or collected and subsequently be used in various ways. For example, the N-terminal fragments can be used as antigens for generating antibodies. The N-terminal fragments can further be used for generating a protein library. In one aspect the invention therefore relates to a protein library comprising multiple different N-terminal fragments obtained by cleavage from the polypeptide of the invention. The library can be used to screen for binding partners, e.g. by mass spectrometry. In another aspect the invention therefore relates to the use of the protein library in a screening method, e.g. in a screening method to identify binding partner of a aGPCR or PC1/PC1-like protein.

### Kits

The present disclosure provides a kit for carrying out a method described herein.

In some cases, a subject kit comprises an expression vector comprising a nucleotide sequence encoding a polypeptide of the present invention.

In some cases, a subject kit comprises a chimeric polypeptide of the present invention.

In some cases, a subject kit comprises a cell that is genetically modified with a nucleic acid comprising a nucleotide sequence encoding a chimeric polypeptide of the present invention.

In some cases, the kit comprises a cell that is genetically modified with a recombinant expression vector comprising a nucleotide sequence encoding the polypeptide of the present invention. Kit components can be in the same container, or in separate containers.

Any of the above-described kits can further include one or more additional reagents, where such additional reagents can be selected from: a dilution buffer; a reconstitution solution; a wash buffer; a control reagent; a control expression vector; a negative control polypeptide (e.g., a chimeric polypeptide that lacks the one or more proteolytic cleavage sites); a positive control polypeptide; a reagent for in vitro production of the chimeric polypeptide, and the like.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic. As such, the instructions may be present in the kits as a package insert, in the labeling of the container of the kit or components thereof. In other embodiments, the instructions are present as an electronic file present on a suitable computer readable storage medium. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### SEQUENCE LISTING

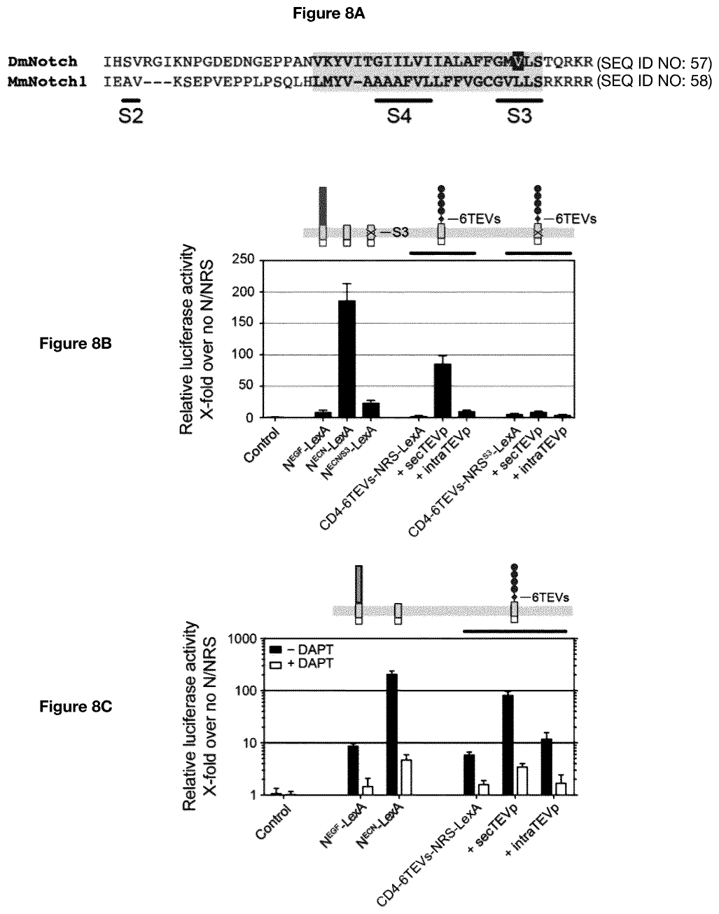

TABLE 2
Overview of the sequences in the sequence listing.

| SEQ ID NO: | Brief description |
|---|---|
| 1 | GAIN domain of human ADGRA2 |
| 2 | GAIN domain of human ADGRA3 |
| 3 | GAIN domain of human ADGRB1 |
| 4 | GAIN domain of human ADGRB2 |
| 5 | GAIN domain of human ADGRB3 |
| 6 | GAIN domain of human ADGRC1 |
| 7 | GAIN domain of human ADGRC2 |
| 8 | GAIN domain of human ADGRC3 |
| 9 | GAIN domain of human ADGRD1 |
| 10 | GAIN domain of human ADGRD2 |
| 11 | GAIN domain of human ADGRE1 |
| 12 | GAIN domain of human ADGRE2 |
| 13 | GAIN domain of human ADGRE3 |
| 14 | GAIN domain of human ADGRE4P |
| 15 | GAIN domain of human ADGRE5 |
| 16 | GAIN domain of human ADGRF1 |
| 17 | GAIN domain of human ADGRF2 |
| 18 | GAIN domain of human ADGRF3 |
| 19 | GAIN domain of human ADGRF4 |
| 20 | GAIN domain of human ADGRF5 |
| 21 | GAIN domain of human ADGRG1 |
| 22 | GAIN domain of human ADGRG2 |
| 23 | GAIN domain of human ADGRG3 |
| 24 | GAIN domain of human ADGRG4 |
| 25 | GAIN domain of human ADGRG5 |
| 26 | GAIN domain of human ADGRG6 |
| 27 | GAIN domain of human ADGRG7 |
| 28 | GAIN domain of human ADGRL1 |
| 29 | GAIN domain of human ADGRL2 |
| 30 | GAIN domain of human ADGRL3 |
| 31 | GAIN domain of human ADGRL4 |
| 32 | GAIN domain of human ADGRV1 |
| 33 | GAIN domain of hPKD1 |
| 34 | GAIN domain of hPKD1L1 |
| 35 | GAIN domain of hPKD1L2 |
| 36 | GAIN domain of hPKD1L3 |
| 37 | GAIN domain of hPKDREJ |
| 38 | GPS/putative tethered agonist segment of C. elegans LAT-1/ADGRL |
| 39 | GPS/putative tethered agonist segment of rat ADGRL1 |
| 40 | GPS/putative tethered agonist segment of D. melanogaster |
| | CIRL/ADGRL |
| 41 | GPS/putative tethered agonist segment of human ADGRG5 |
| 42 | GPS/putative tethered agonist segment of human ADGRG1 |
| 43 | GPS/putative tethered agonist segment of human ADGRD1 |
| 44 | GPS/putative tethered agonist segment of human ADGRE5 |
| 45 | GPS/putative tethered agonist segment of human ADGRG6 |
| 46 | GPS/putative tethered agonist segment of D. melanogaster |
| | FMI/ADGRC |
| 47 | GPS/putative tethered agonist segment of human PC1 |
| 48 | GPS/putative tethered agonist segment of C. elegans LOV-1/PC1 |
| 49 | GPS/putative tethered agonist segment of human PC1L1 |
| 50 | GPS/putative tethered agonist segment of human PC1L2 |
| 51 | GPS/putative tethered agonist segment of human PC1L3 |
| 52 | Sequence comprising human Notch1 iuxta- and transmembrane |
| | segment |
| 53 | Sequence comprising human Notch2 iuxta- and transmembrane |
| | segment |
| 54 | Sequence comprising human Notch3 iuxta- and transmembrane |
| | segment |
| 55 | Sequence comprising human Notch4 iuxta- and transmembrane |
| | segment |
| 56 | Sequence comprising drosophila melanogaster Notch iuxta- and |
| | transmembrane segment |
| 57 | Drosophila melanogaster Notch iuxta- and transmembrane segment |
| | (FIG. 8) |
| 58 | mouse Notch1 iuxta- and transmembrane segment (FIG. 8) |
| 59 | Full-length NTF release sensor for dmCIRL |
| 60 | Full-length NTF release sensor for dmCIRL (GPS cleavage site |
| | mutated) |
| 61 | Full-length NTF release sensor for dmCIRL (S3 cleavage site |
| | mutated) |
| 62 | NTF release sensor positive control |
| 63 | NTF release sensor negative control |

### EXAMPLES

Operating Principle of the NRS for Adhesion GPCRs and PC1/PC1-Like Molecules

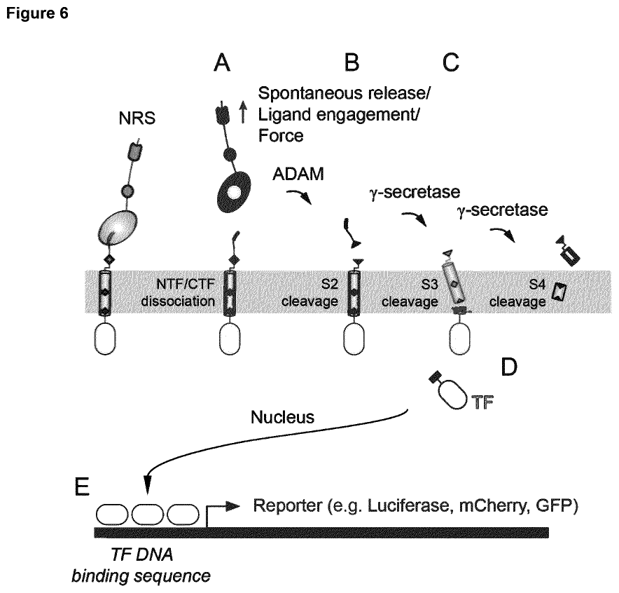

We have harnessed the regulated intramembrane proteolysis (RIP) principle employed in Notch receptor activation for the NRS. Notch is the key element in an evolutionarily highly conserved pathway that controls binary developmental decisions and plays essential roles in the determination of cell lineages (reviewed in ref.²⁴). Briefly, Notch is activated through one of its ligands Delta or Serrate, which are transmembrane proteins mounted in cis and in trans to the receptor protein. Ligand endocytosis in trans transmits mechanical force onto the Notch ECR (extracellular region) causing the exposure of a iuxtamembrane receptor region^{25,26}, which during signal inactivity is shielded by the NRR (negative regulatory region). Consequently, the iuxtamembrane S2 site becomes accessible to the matrix metalloprotease Kuzbanian/ADAM (a disintegrin and metalloprotease domain), which cleaves the receptor protein outside the plasma membrane leading to shedding of a large part of the Notch ECR. The working principle of the NRS (FIG. 6) relies on (J) GAIN domain autoproteolysis or proteolysis of other domains in the ECR of aGPCR/PC1/PC1-like protein, and the coupling of (ii) NTF-CTF separation at the GPS and subsequent NTF release and exposure of its S2 site, (iii) further proteolytic processing of the Notch^{ITS} component, to the (iv) intracellular release of a transcription factor moiety, which leads to the transcriptional activation of a genetic reporter element whose expression can be quantified by means of standard biochemical or optical assay systems.

### NTF-CTF Separation at the GPS and Subsequent NTF Release Off NRS and S2 Site Exposure of NRS

In the naturally occurring setting for Notch signaling its S2 proteolysis site is protected by multiple EGF domain repeats, which mediate physiological ligand interactions, and the NNR (Notch-1 negative regulatory) region of the receptor's extracellular part of the receptor (reviewed in ref.²⁷). EGF repeats and the NRR can be entirely replaced by heterologous domains while still protecting the signal-initiating S2 site from Kuz/ADAM engagement²⁸ (FIG. 6). Accordingly, in case of the NRS activation at the S2 site is suppressed by aGPCR/PC1/PC1-like molecule extracellular regions.

NTF-CTF separation within the NRS is made possible through posttranslational GAIN domain autoproteolysis and subsequent covalent stabilisation of the ensuing NTF-CTF heterodimer or similar autoproteolytic events in other extracellular domains of aGPCRs/PC1/PC1-like proteins. NTF-CTF separation is induced spontaneously, through ligand binding, mechanical stimulation or a combination of the aforementioned processes (FIG. 6, Step A). As a result of NTF release, the remaining portion of the NRS consisting of the aGPCR/PC1/PC1-like^{TA}-Notch^{ITS}-TF chimeric protein is left in the plasma membrane with the S2 site of the Notch^{ITS} exposed and accessible for subsequent proteolytic attack.

Proteolytic Processing of Iuxta- and Transmembrane Segment of NRS

S2 proteolytic processing of the NRS occurs through ADAM-like matrix metalloproteinases present in the test cell (FIG. 6, Step B). This allows for successive proteolytic steps of the remaining Notch^{ITS} at S3 and S4 sites, which are located within the NRS' single transmembrane helix, through the γ-secretase complex (FIG. 6, Step C) in a process termed regulated intramembrane proteolysis (RIP)²⁹⁻³¹

Intracellular Release of Transcription Factor Moiety

The Notch intracellular domain (NICD) was replaced with a heterologous transcription factor, (LexA::VP16 cassette), so that NRS activation upon NTF release results in the liberation of the TF from the intracellular face of the membrane (FIG. 6, Step D), which in turn and can be read out through the LexA/lexAop binary expression system²¹ (FIG. 6, Step E). This biosynthetic replacement of the NICD has been similarly employed in other biosensors previously^{30,32,33}

### Example 1: Demonstration of NRS Functionality

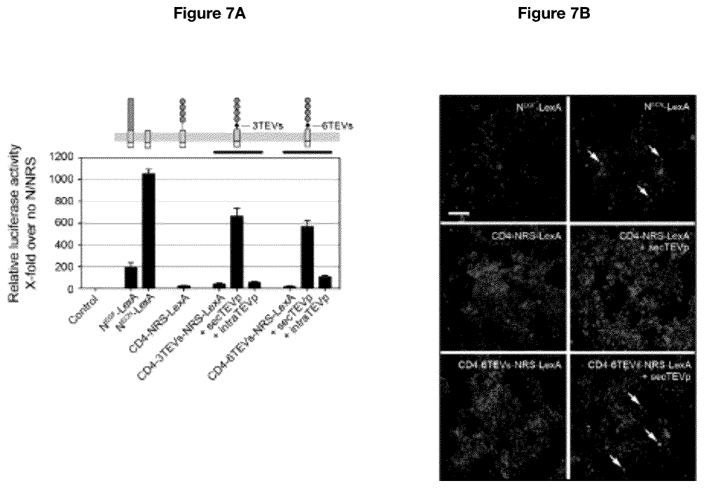

In order to conceptually establish the approach to fuse heterologous ECRs to the Notch^{ITS} to suppress its proteolytic activation, a sensor protein containing 4 immunoglobulin (Ig) domains of the human CD4 receptor fused to the Notch^{ITS} was engineered²⁸ (FIG. 7A). In a luciferase assay in Drosophila Schneider 2 (S2) cells addition of the CD4 domains to the NRS-LexA sensor component sufficiently quenched LexA release comparable to a constitutively inactive N^{EGF}-LexA control as previously used in Drosophila melanogaster³⁴ (FIG. 7A).

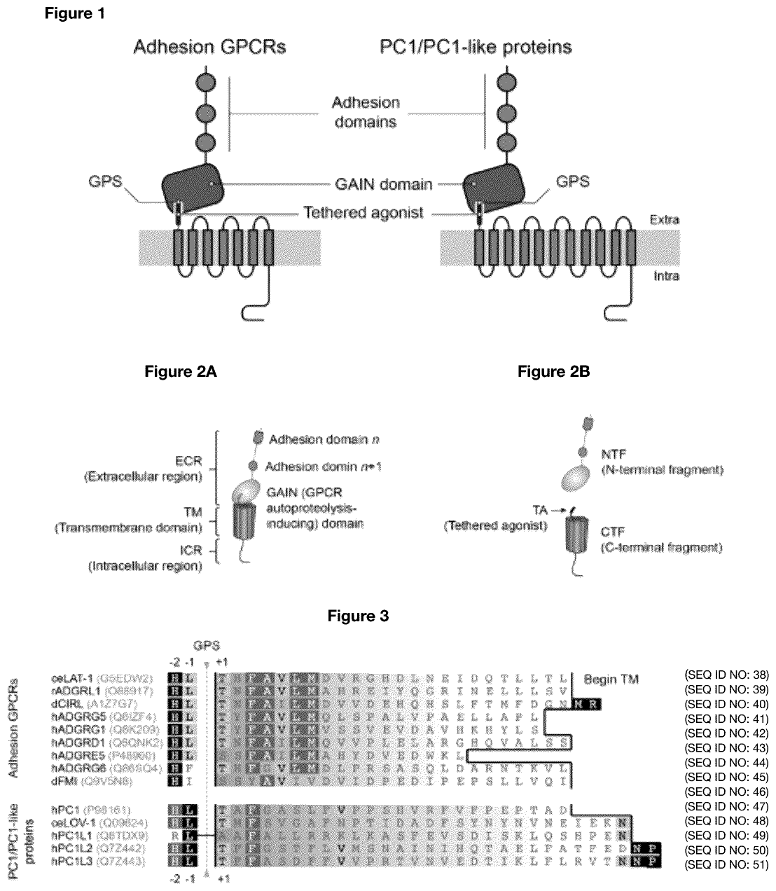

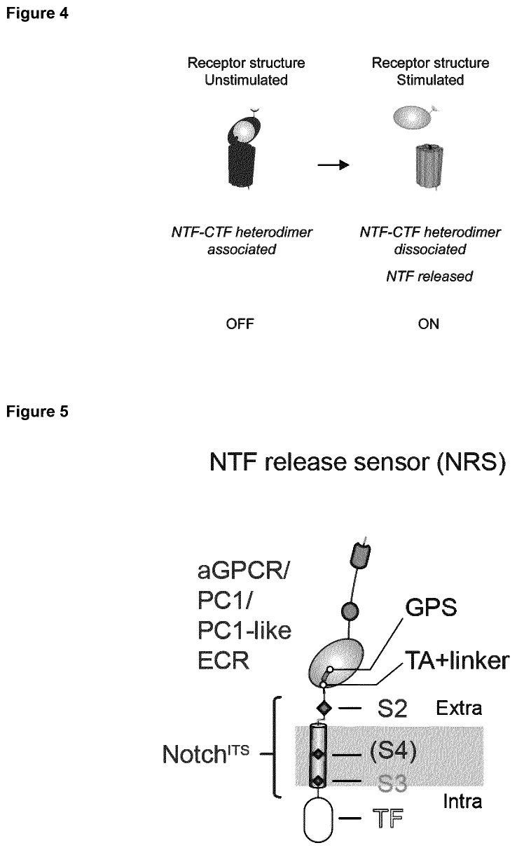

A tri- or hexarepeat of the highly selective TEV (Tobacco etch virus) protease cleavage site (TEVs) was introduced between CD4 repeats and ITS (CD4-3TEVs-NRS-LexA, CD4-6TEVs-NRS-LexA) in order to conditionally sever artificial ECR and Notch^{ITS} of the sensor and expose the extracellular S2 site by TEV protease expression. Upon co-expression of a secreted version of TEV (secTEVp), but not when TEV was expressed intracellularly (intraTEVp) or not at all, strong activation of both CD4-3/6TEVs-NRS-LexA reporters were observed in S2 cells by a commercially available firefly luciferase lexAop-FLuc reporter (FIG. 2A), and through expression of a lexAop-mCherry reporter (FIG. 7A), co-transfected with the sensors in S2 cells, respectively (FIG. 7B). As a positive control a constitutively active N^{ECN}-LexA receptor³⁴ with a constantly exposed S2 cleavage site was included (FIG. 7A,B).

Replacement of a critical Valin residue in the S3 site of Notch^{ITS} by Lysine²⁹ (FIG. 8A,B), and pharmacological inhibition of γ-secretase activity by 10 μM DAPT abolished reporter activity (FIG. 8C), demonstrating that NRS activation is relayed through the canonical Notch proteolytic cascade and that the readout of NRS activation can be pharmacologically modulated.

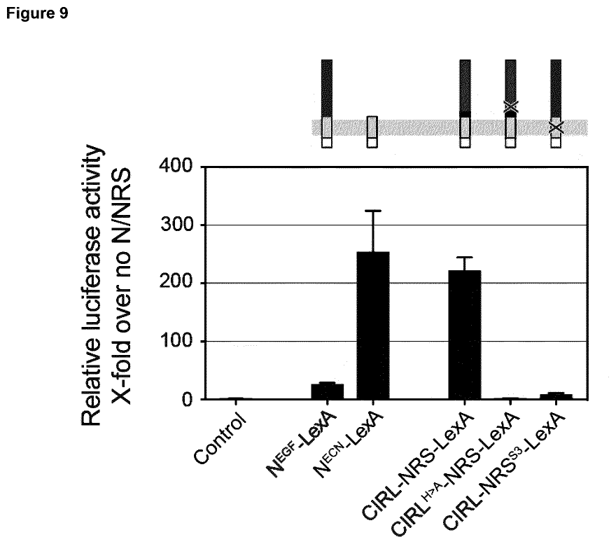

Next, the extracellular CD4 repeats of the sensor protein were replaced with the ECR of an aGPCR, the Latrophilin/ADGRL-homolog CIRL/CG8639 (calcium independent receptor of α-latrotoxin) to enable the NRS to detect the physical dissociation of the NTF-CTF complex within the GAIN domain when appended to the Notch^{ITS}. Thus, the entire ECR of CIRL (including the GAIN domain, GPS, tethered agonist segment and predicted linker to the 7TM domain) was N-terminally fused to the NRS core (CIRL-NRS-LexA; FIG. 9). In luciferase assays we noticed that CIRL-NRS-LexA readily showed high activity suggesting that in this sensor layout either the S2 site was not sufficiently protected from the proteolytic attack of the metalloprotease by the CIRL-ECR, or that the NTF-CTF heterodimer spontaneously dissociated under the assay conditions rendering the S2 site exposed (FIG. 9). In order to distinguish between these two possibilities, we mutated the GPS of the sensor at the −2 position of its catalytic triad and replaced the conserved Histidin by an Alanine (FIG. 9) to block GAIN domain self-cleavage and eventual NTF release. Importantly, CIRL^{H>A}-NRS-LexA displayed no reporter activity in luciferase assays similar to a CIRL-NRS^{S3}-LexA sensor (disabled γ-secretase recognition) and mock transfected cells (FIG. 9). This set of experiments establishes that the CIRL-NRS-LexA NRS can be used for monitoring the integrity of the NTF-CTF heterodimer clamped by the GAIN domain of CIRL.

### Example 2: Demonstration of NRS Functionality in Drosophila melanogaster

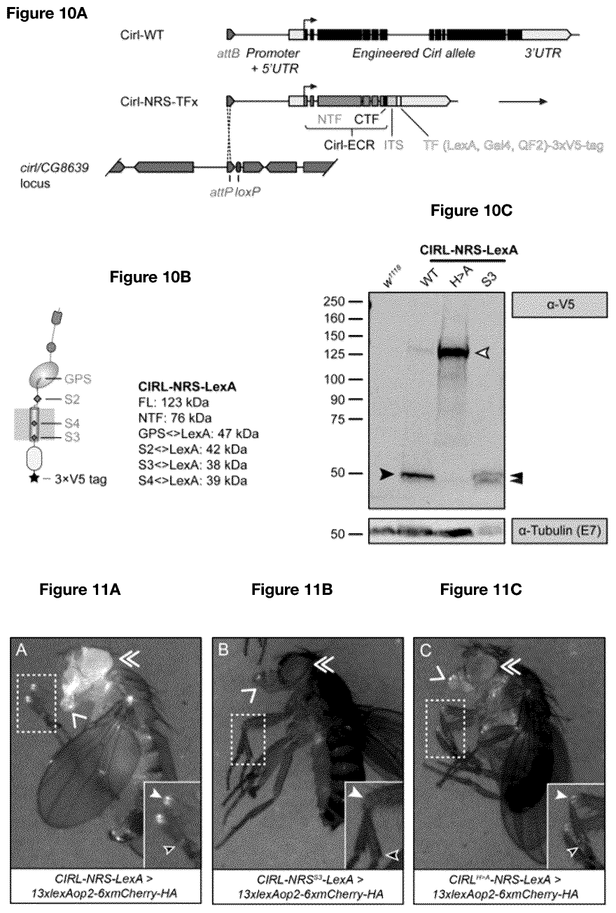

To test the utility of the NRS sensor principle in vivo, an existing genomic engineering platform^{13,35} was used to encode NRS sensors in alleles of the adhesion GPCR cirl/CG8639 of Drosophila melanogaster. This places CIRL-NRS expression under the endogenous regulatory control of the cirl locus (FIG. 10A). The NRS design was adapted to generate minigenes for cirl-NRS-LexA, cirl^{H>A}-NRS-LexA and cirl-NRS^{S3}-LexA by fusing the genomic fragment encoding the CIRL-ECR to the NRS-LexA cDNA (FIG. 10A,B). The resulting transgenes were inserted in the cirl gene using phiC31-assisted transformation generating fly stocks expressing the different sensors variants from the cirl locus.

Western blot analysis of fly head homogenates expressing CIRL-NRS-LexA and CIRL-NRS^{S3}-LexA confirmed that S3 proteolytic processing of CIRL-NRS-LexA is quenched by the mutation (FIG. 10C). Further, protein levels of CIRL-NRS-LexA and CIRL^{H>A}-NRS-LexA were indistinguishable, and GPS cleavage was entirely blocked by the GPS mutation resulting in a single full-length protein band running at the predicted size of 123 kDa (FIG. 10C). Importantly, no further CIRL-NRS-LexA fragments derived from S2/S3/S4 cleavages were observed (FIG. 10C). This demonstrates that CIRL-NRS-LexA activity is absolutely dependent on NTF release, and that the chain of molecular events allowing for the detection of NTF release through the NRS is unidirectional, and sequentially unfolds in the intended order: NTF/CTF disruption →S2 cleavage →S3 cleavage →LexA release (FIG. 10B).

Next, the functionality of CIRL-NRS was further studied in anatomical expression analyses. Expression of cirl-NRS-LexA in thirs instar larvae showed that the CIRL-NRS-LexA is activated only in Cirl⁺ neurons in the peripheral nervous system, the ventral nerve cord (VNC) and the brain hemispheres such as previously through a transcriptional cirlp-Gal4 reporter³⁵ (data not shown). In adult animals cirl-NRS-LexA expression recapitulates endogenous cirl expression³⁵ with strong expression in the eye, proboscis and leg neurons (FIG. 11A). Inhibition of S3 cleavage through the V→K mutation completely abrogated CIRL-NRS^{S3}-LexA sensor activity apparent by lack of lexAop-mCherry expression at all major expression sites (eye, leg joints, proboscis; FIG. 11B), indicating that intracellular LexA release is critically dependent on NRS processing through events that precede γ-secretase cleavage in vivo too.

Disabling GAIN domain proteolysis and thus potential NTF release of CIRL through replacement of a critical Histidin residue at position −2 to the GPS of the CIRL-NRS-LexA protein (CIRL^{H>A}-NRS-LexA) abrogated NRS activation apparent through loss of the mCherry reporter induction in all organs and tissues (FIG. 11C).

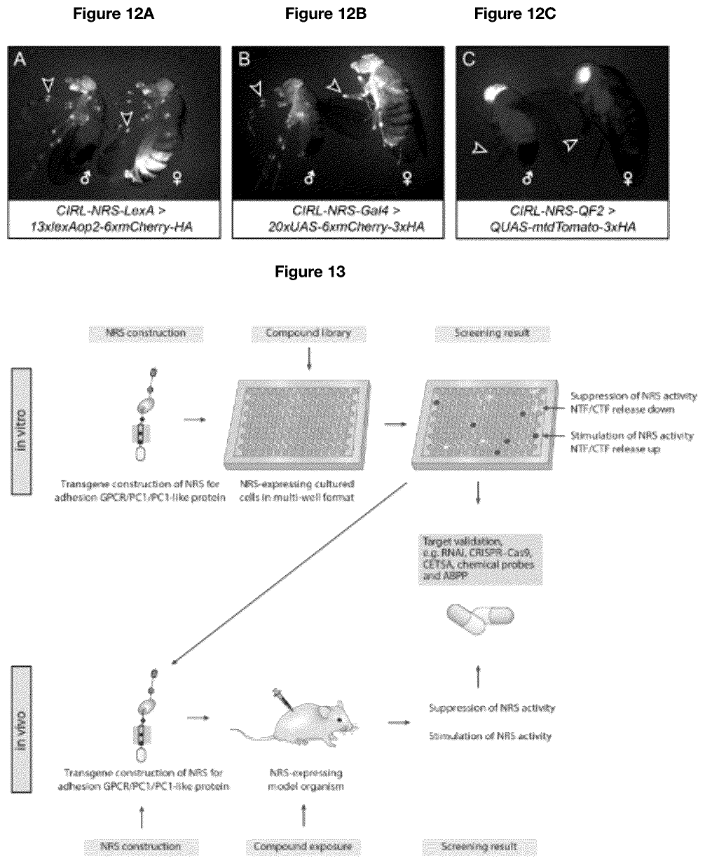

To control for potential misexpression inherent to the LexA transcriptional reporter, additional cirl-NRS transgenes alternatively terminating in Ga/4 or QF2 transcription factor cassettes were generated an analysed. A comparison of expression patterns controlled by cirl-NRS-LexA (FIG. 12A), cirl-NRS-Ga/4 (FIG. 12A) and cirl-NRS-QF2 (FIG. 12A) in adult animals using matching reporter transgenes displayed strong activation of the first two NRS variants (FIG. 12A,B). cirl-NRS-QF2 showed a weaker yet anatomically similar pattern (FIG. 12C).

Amino acid sequences:
SEQ ID NO:59. Full-length NTF release sensor for dmCIRL
SEQ ID NO:60 Full-length NTF release sensor for dmCIRL (GPS cleavage site mutated)
SEQ ID NO:61 Full-length NTF release sensor for dmCIRL (S3 cleavage site mutated)
SEQ ID NO:62 NTF release sensor positive control
SEQ ID NO:63 NTF release sensor negative control

### Materials and Methods

### S2 Cell Culture

For transfection, mini- or midi-prepped plasmid DNA (Qiagen) for each construct was adjusted to a stock concentration 100 ng/μl using a nanophotometer (Implen). For individual transfections a total amount of 200 ng DNA per 96-well plate well was used and always contained 30 ng of lexAop>FLuc2 (test construct reporter) and Act5.1Cp>RLuc (transfection control reporter), respectively. Test constructs (X-NRS-LexA) and TEV protease plasmids were added at 1:1 ratio (equimolarly adjusted to lexAop>FLuc2) to the DNA mixes; each DNA mix was supplemented with empty pBSK-SK+ vector (Stratagene) to 200 ng. S2 cells were cultured in Schneider's Drosophila medium (Invitrogen, Cat. no. 21720-024). Cultures were maintained in an air incubator at 25° C. S2 cells were split and plated into individual wells of a 96-well plate at a concentration of 5×10⁵/well on day 0. 24 hours after plating (day 1), cells were transfected with Lipofectamine 2000 (Invitrogen, #11668019) with the suitable 1:1 plasmid/reagent mixture according to manufacturer's instructions and incubated for 48 hours. When induction of the metallothionein-promoter was required, CuSO₄ stock medium was added in each well of the experiment to a final concentration of 0.5 mM CuSO₄ 24 hrs after transfection (day 2).

### Luciferase Assays

Cells were lysed on day 3 (48 hrs after transfection) and luciferase measurements with the Dual-Glo luciferase assay system (Promega) were performed according to the manufacturer's protocol. In brief, the supernatant was removed from each well by aspiration and cells were incubated with 200 μl passive lysis buffer on a shaker at room temperature for 30 min. Lysates were analyzed in 96-well plates with a Victor2 plate reader luminometer (PerkinElmer). Firefly and Renilla luciferase luminescence signals were collected for 10 s. Relative luciferase activities were calculated for each sample individually as principally described in Potter et al. (ref. 23) according to the following formula:

RLAₓ=(Fₓ/Rₓ)/(F̅/̅R̅)_{Empty}

where

(F̅/̅R̅)_{Empty}=(Σᵢ₌₁ⁿF_{Empty}ⁱ/R_{Empty}ⁱ)/n,

where n=number of control samples; F=Firefly luciferase luminescence signal; R=Renilla luciferase luminescence signal; Empty=lexAop>FLuc2+Ac5.1p>RLuc+BSK-SK+. Each condition was executed at least in triplicate, the average and SEM were determined for each condition, and statistical significance was evaluated using Student's t test. Plasmids used for transfection in S2 cells were: N^{ECN}-LexA, N^{EGF}-LexA, ChiN-Test, pChiN1, pChiN2, pChiN3, lexAop-FLuc2, Ac5.1-RLuc, secTEVp, intraTEVp, pBSK-SK+.

### Immunoblots

Fly heads were collected into 0.5 ml Eppi and immediately frozen in liquid nitrogen. Next, heads were mechanically crushed in 40 μl SDS (2%, supplemented with protease Inhibitor Cocktail, Sigma-Aldrich; 1:1000) using a glass stirrer. Samples were incubated on ice for 10 min before the addition of 4 μl Triton-X100 (10%). Next, SDS-based sample buffer (Li-cor) was supplemented with β-Mercaptoethanol and was added to final dilution of 1×. Samples were centrifuged for 30 min at 14000 rpm (4° C.) and supernatant was collected. Centrifugation step was repeated, supernatant collected in fresh tube and subjected to electrophoresis on 6% or 4-12% Tris-Glycin SDS gel (Novex-Wedge-Well; Invitrogen) and blotted onto nitrocellulose membrane (0.2 μm pore size). The membrane was blocked for 1 hour (RT) using Odyssey Blocking buffer (Li-cor) diluted 1:2 with 1×PBS.

Blots were probed with primary antisera at the indicated concentrations overnight at 4° C.: rabbit-α-HA (1:1000, RRID: AB_10693385), mouse-α-V5 (1:500; RRID:AB_2556564), mouse-α-tubulinβ (1:5000, RRID:AB_528499), rabbit-α-tubulinα (1:500, RRID:AB_25541125). After rinsing twice and 3-10 min washing steps, membranes were incubated with IRDye 680RD goat-α-rabbit (RRID:AB_2721181) and goat-α-mouse (RRID:AB_2651128) as well as 800CW goat-α-mouse (1:15000; RRID:AB_2687825) and goat-α-rabbit (1:15000; RRID:AB_2651127) for 1 hour at RT, and again rinsed twice and washed 3-10 min. Blots were imaged with an OdysseyFc 2800 (Li-cor).

### REFERENCES

1. Rask-Andersen, M., Almen, M. S. & Schioth, H. B. Trends in the exploitation of novel drug targets. Nature reviews Drug discovery 10, 579-590 (2011).
2. O'Hayre, M. et al. The emerging mutational landscape of G proteins and G-protein-coupled receptors in cancer. Nat. Rev. Cancer 13, 412-424 (2013).
3. Promel, S., Langenhan, T. & Arac, D. Matching structure with function: the GAIN domain of adhesion-GPCR and PKD1-like proteins. Trends Pharmacol. Sci. 34, 470-478 (2013).
4. Langenhan, T., Aust, G. & Hamann, J. Sticky signaling—adhesion class G protein-coupled receptors take the stage. Science signaling 6, re3 (2013).
5. Scholz, N., Monk, K. R., Kittel, R. J. & Langenhan, T. Adhesion GPCRs as a Putative Class of Metabotropic Mechanosensors. Handb Exp Pharmacol 234, 221-247 (2016).
6. Purcell, R. H. & Hall, R. A. Adhesion G Protein-Coupled Receptors as Drug Targets. Annu. Rev. Pharmacal. Toxicol. 58, 429-449 (2018).
7. Langenhan, T. Adhesion G protein-coupled receptors-Candidate metabotropic mechanosensors and novel drug targets. Basic Clin. Pharmacal. Toxicol. 547, 145 (2019).
8. Langenhan, T., Piao, X. & Monk, K. R. Adhesion G protein-coupled receptors in nervous system development and disease. Nat Rev Neurosci 17, 550-561 (2016).
9. Arac, D. et al. A novel evolutionarily conserved domain of cell-adhesion GPCRs mediates autoproteolysis. EMBO J 31, 1364-1378 (2012).
10. Lin, H.-H. et al. Autocatalytic cleavage of the EMR2 receptor occurs at a conserved G protein-coupled receptor proteolytic site motif. J Biol Chem 279, 31823-31832 (2004).
11. Krasnoperov, V. G. et al. alpha-Latrotoxin stimulates exocytosis by the interaction with a neuronal G-protein-coupled receptor. Neuron 18, 925-937 (1997).
12. Gray, J. X. et al. CD97 is a processed, seven-transmembrane, heterodimeric receptor associated with inflammation. J Immunol 151, 5438-5447 (1996).
13. Scholz, N. et al. Mechano-dependent signaling by Latrophilin/CIRL quenches cAMP in proprioceptive neurons. elife 6, 1364 (2017).
14. Promel, S. et al. Characterization and functional study of a cluster of four highly conserved orphan adhesion-GPCR in mouse. Dev Dyn 241, 1591-1602 (2012).
15. Promel, S. et al. The GPS Motifls a Molecular Switch for Bimodal Activities of Adhesion Class G Protein-Coupled Receptors. Cell Reports 2, 321-331 (2012).
16. Nieberler, M., Kittel, R. J., Petrenko, A. G., Lin, H.-H. & Langenhan, T. Control of Adhesion GPCR Function Through Proteolytic Processing. Handb Exp Pharmacol 234, 83-109 (2016).
17. Paavola, K. J., Stephenson, J. R., Ritter, S. L., Alter, S. P. & Hall, R. A. The N terminus of the adhesion G protein-coupled receptor GPR56 controls receptor signaling activity. Journal of Biological Chemistry 286, 28914-28921 (2011).
18. Liebscher, I. et al. A Tethered Agonist within the Ectodomain Activates the Adhesion G Protein-Coupled Receptors GPR126 and GPR133. Cell Reports 9, 2018-2026 (2014).
19. Stoveken, H. M., Hajduczok, A. G., Xu, L. & Tall, G. G. Adhesion G protein-coupled receptors are activated by exposure of a cryptic tethered agonist. Proc Natl Acad Sci USA 112, 6194-6199 (2015).
20. Liebscher, I. & Schöneberg, T. Tethered Agonism: A Common Activation Mechanism of Adhesion GPCRs. Handb Exp Pharmacol 234, 111-125 (2016).
21. Lai, S.-L. & Lee, T. Genetic mosaic with dual binary transcriptional systems in Drosophila. Nat Neurosci 9, 703-709 (2006).
22. Brand, A. H. & Perrimon, N. Targeted gene expression as a means of altering cell fates and generating dominant phenotypes. Development 118, 401-415 (1993).
23. Potter, C. J., Tasic, B., Russler, E. V., Liang, L. & Luo, L. The Q system: a repressible binary system for transgene expression, lineage tracing, and mosaic analysis. Cell 141, 536-548 (2010); Riabinina O, Luginbuhl D, Marr E, Liu S, Wu M N, Luo L, Potter CJ. Improved and expanded Q-system reagents for genetic manipulations”. Nature Methods. 12 (3): 219-22, 5 p following 222 (2015).
24. Kopan, R. & Ilagan, M. X. G. The canonical Notch signaling pathway: unfolding the activation mechanism. Cell 137, 216-233 (2009).
25. Stephenson, N. L. & Avis, J. M. Direct observation of proteolytic cleavage at the S2 site upon forced unfolding of the Notch negative regulatory region. Proc Natl Acad Sci USA 109, E2757-65 (2012).
26. Meloty-Kapella, L., Shergill, B., Kuon, J., Botvinick, E. & Weinmaster, G. Notch ligand endocytosis generates mechanical pulling force dependent on dynamin, epsins, and actin. Dev Cell 22, 1299-1312 (2012).
27. Gordon, W. R., Arnett, K. L. & Blacklow, S. C. The molecular logic of Notch signaling: a structural and biochemical perspective. J Cell Sci 121, 3109-3119 (2008).
28. Mumm, J. S. et al. A ligand-induced extracellular cleavage regulates gamma-secretase-like proteolytic activation of Notch1. Mol Cell 5, 197-206 (2000).
29. Schroeter, E. H., Kisslinger, J. A. & Kopan, R. Notch-1 signalling requires ligand-induced proteolytic release of intracellular domain. Nature 393, 382-386 (1998).
30. Struhl, G. & Adachi, A. Nuclear access and action of notch in vivo. Cell 93, 649-660 (1998).
31. Kopan, R., Schroeter, E. H., Weintraub, H. & Nye, J. S. Signal transduction by activatedmNotch: importance of proteolytic processing and its regulation by the extracellular domain. Proc Natl Acad Sci USA 93, 1683-1688 (1996).
32. Vooijs, M. et al. Mapping the consequence of Notch1 proteolysis in vivo with NIP-CRE. Development 134, 535-544 (2007).
33. Morsut, L. et al. Engineering Customized Cell Sensing and Response Behaviors Using Synthetic Notch Receptors. Cell 164, 780-791 (2016).
34. Struhl, G. & Adachi, A. Requirements for presenilin-dependent cleavage of notch and other transmembrane proteins. Mol Cell 6, 625-636 (2000).
35. Scholz, N. et al. The Adhesion GPCR Latrophilin/CIRL Shapes Mechanosensation. Cell Reports 11, 866-874 (2015).
36. Gresko, N. et al. Polycystin 1 is an atypical adhesion GPCR that responds to non-canonical WNT signals and inhibits GSK3β. The FASEB Journal 33.1-supplement 863-10 (2019).
37. Harmar, A. J. Family-B G-protein-coupled receptors. Genome biology 2(12), reviews 3013-1 (2001).
38. Salzman G S. et al. Structural Basis for Regulation of GPR56/ADGRG1 by Its Alternatively Spliced Extracellular Domains. Neuron 91, 1292-1304 (2016).
39. Leon K. et al. Structural basis for adhesion G protein-coupled receptor Gpr126 function. Nature Communications 11, 194 (2020).
40. Abe, J., Fukuzawa, T. & Hirose, S. Cleavage of Ig-Hepta at a “SEA” module and at a conserved G protein-coupled receptor proteolytic site. The Journal of Biological Chemistry 277, 23391-23398 (2002). https://doi.org/10.1074/jbc.M110877200
41. Fukuzawa, T. & Hirose, S. Multiple processing of Ig-Hepta/GPR116, a G protein-coupled receptor with immunoglobulin (Ig)-like repeats, and generation of EGF2-like fragment. Journal of Biochemistry 140, 445-452 (2006).
42. Krasnoperov, V. et al. Dissociation of the Subunits of the Calcium-Independent Receptor of α-Latrotoxin as a Result of Two-Step Proteolysis. Biochemistry 48, 3230-3238 (2009).
43. Cork, S. M. et al. A proprotein convertase/MMP-14 proteolytic cascade releases a novel 40 kDa vasculostatin from tumor suppressor BA11. Oncogene 31, 5144-5152 (2012).
44. Moriguchi, T. et al. DREG, a developmentally regulated G protein-coupled receptor containing two conserved proteolytic cleavage sites. Genes Cells 9, 549-560 (2004).
45. Okajima, D., Kudo, G. & Yokota, H. Brain-specific angiogenesis inhibitor 2 (BAI2) may be activated by proteolytic processing. J Recept Sig Transd 30, 143-153 (2010).
46. Kaur, B., Brat, D. J., Devi, N. S. & Meir, E. G. V. Vasculostatin, a proteolytic fragment of Brain Angiogenesis Inhibitor 1, is an antiangiogenic and antitumorigenic factor. Oncogene 24, 3632-3642 (2005).

1. A polypeptide comprising: (i) a first sequence comprising a GPCR autoproteolysis-inducing (GAIN) domain of (a) an adhesion GPCR or of (b) PC1/PC1-like protein, (ii) a second sequence comprising the transmembrane region of a Notch receptor, and (iii) a third sequence comprising a transcription factor moiety.

2. The polypeptide of claim 1, wherein release of an N-terminal portion of the first sequence at one or more proteolytic cleavage sites induces cleavage of the second sequence, thereby releasing the transcription factor moiety.

3. The polypeptide of claim 2, wherein said release of the N-terminal portion requires proteolysis at a GPCR proteolysis site (GPS) and/or triggered by proteolysis at a GPCR proteolysis site (GPS) within the GAIN domain.

4. The polypeptide of claim 1, wherein the first sequence comprises an N-terminal part of said adhesion GPCR or of said PC1/PC1-like protein, from the N-terminal amino acid of the aGPCR or the PC1/PC1-like protein to the C-terminal end of the GAIN domain of the aGPCR or the PC1/PC1-like protein.

5. The polypeptide of claim 1, wherein the first sequence comprises an extracellular region (ECR) of said aGPCR or said PC1/PC1-like protein, such that the first sequence of the polypeptide ends before the start of transmembrane (TM) domain of the aGPCR or the PC1/PC1-like protein.

6. The polypeptide of claim 1, wherein the first sequence comprises a fragment of an extracellular region (ECR) of the aGPCR or the PC1/PC1-like protein, wherein said fragment comprises said GAIN domain.

7. The polypeptide of claim 1, wherein the first sequence comprises: (a) an amino acid sequence selected from the group consisting of SEQ ID NOs:1-32, (b) an amino acid which has a sequence identity of at least 90% to any one of SEQ ID NOs:1-32, or (c) an amino acid sequence which differs from an amino acid sequence as shown in any one of SEQ ID NOs:1-32 by less than 50 amino acids.

8. The polypeptide of claim 1, wherein the second sequence comprises an S2 cleavage site and an S3 cleavage site of the Notch receptor.

9. The polypeptide of claim 8, wherein the second sequence further comprises an S4 cleavage site of the Notch receptor.

10. The polypeptide of claim 1, wherein said polypeptide comprises an amino acid sequence as shown in SEQ ID NO:59, or an amino acid sequence having a sequence identity of at least 90% to SEQ ID NO: 59.

11. The polypeptide of any claim 1, wherein said polypeptide is a is a sensor protein suitable for detecting the release of an N-terminal fragment and/or proteolytic cleavage within the GAIN domain.

12. A nucleic acid encoding the polypeptide of claim 1.

13. A plasmid or vector comprising the nucleic acid of claim 12.

14. A cell comprising the polypeptide of claim 1, a nucleic acid encoding said polypeptide, or a plasmid or vector comprising a nucleic acid encoding said polypeptide in combination with nucleic acid capable of binding to the transcription factor moiety, operably linked to a nucleic acid encoding a reporter.

15. The cell of claim 14, wherein said transcription factor moiety, upon release from the second sequence, is capable of inducing expression of the reporter.

16. A non-human transgenic animal comprising the polypeptide of claim 1, a nucleic acid encoding said polypeptide, a plasmid or vector comprising a nucleic acid encoding said polypeptide, or a cell comprising said polypeptide, said nucleic acid, said plasmid or said vector.

17. A screening method comprising the steps of:
a. contacting a test compound with the cell of claim 14, and
b. determining the level of the reporter or of a detectable signal caused by said reporter.

18. (canceled)

19. A screening method comprising the steps of:
a. administering a test compound to the non-human transgenic animal of claim 16, and
b. determining the level of a reporter or of a detectable signal caused by said reporter.

20. A non-human transgenic animal expressing the polypeptide of claim 1.

21. The polypeptide of claim 1, wherein the first sequence comprises an amino acid sequence which differs from an amino acid sequence as shown in any one of SEQ ID NOs:1-32 by less than 3-10 amino acids.
